(19) United States
(12) Patent Application Publication
(10) Pub. No.: US 2025/0185668 A1
(43) Pub. Date: Jun. 12, 2025

(54) BACTERIOPHAGE, BACTERIAL WILT DISEASE CONTROL AGENT, AND BACTERIAL WILT DISEASE CONTROL METHOD

(71) Applicant: PANEFRI INDUSTRIAL CO., LTD., Nagaokakyo-shi (JP)

(72) Inventors: Toshiaki HARASHIMA, Okinawa (JP); Kazuma NAKANO, Okinawa (JP); Toru KARIMATA, Okinawa (JP); Kumiko HOKAMA, Okinawa (JP)

(73) Assignee: PANEFRI INDUSTRIAL CO., LTD., Nagaokakyo-shi (JP)

(21) Appl. No.: 18/007,432

(22) PCT Filed: Jul. 30, 2020
(86) PCT No.: PCT/JP2020/029189

§ 371 Date: Jan. 30, 2023

(51) Int. Cl. A01N 63/40; A01P 1/00; C07K 14/005; C12N 7/00

(52) U.S. Cl. CPC A01N 63/40; A01P 1/00; C07K 14/005; C12N 7/00; C12N 2795/10221; C12N 2795/10222; C12N 2795/10232; C12N 2795/10271

(57) ABSTRACT

Bacteriophages that comprise a tail fiber protein sequence comprising a C terminus host recognition site described by SEQ ID NO: 1 and which infect Ralstonia solanacearum which causes bacterial wilt disease are disclosed. Compositions comprising these bacteriophages may be employed as wilt control agents. Methods for preventing or treating diseases caused by Ralstonia solanacearum such as bacterial wilt disease are also disclosed. A bacterial wilt disease control method in a plant may include administering such bacteriophages to a plant or to a plant growth medium and can be effective against a wide variety of Ralstonia solanacearum strains. Nucleic acids encoding the C terminus host recognition site and amino acid sequences comprising the host recognition site are also disclosed.

### CROSS-REFERENCE TO RELATED APPLICATIONS

The present application is the national stage of international application PCT/JP2020/029189, filed on Jul. 30, 2020.

### TECHNICAL FIELD

The present invention relates to a bacteriophage, a bacterial wilt disease control agent and a bacterial wilt disease control method.

### BACKGROUND ART

Ralstonia solanacearum infects 200 plant species or more such as the Solanaceae plants and causes bacterial wilt. When the disease progresses, the plant infected with Ralstonia solanacearum withers.

Major chemical pesticides which have been used so far against bacterial wilt are chloropicrin and bromomethane, which are powerful drugs.

In view of the problems such as the increase in the effective application amounts, the environmental pollution, the ozone layer depletion, the influences on the health and the pesticide residues, however, development of safe alternative pesticides and control techniques which replace the chemical pesticides is desired.

Although techniques using bacteriophages (PTLs 1 to 3) have been known so far as safe alternative pesticides and control techniques which replace the chemical pesticides for example, the techniques are effective against a limited number of Ralstonia solanacearum strains, and a technique which is effective against a wider variety of Ralstonia solanacearum strains has been desired.

### CITATION LIST

### Patent Literature

PTL 1: JP-A-2007-252351
PTL 2: JP-A-2018-24589
PTL 3: WO2017/104347

### SUMMARY OF INVENTION

### Technical Problem

Accordingly, a problem of the invention is to provide a bacteriophage which is effective against a wider variety of Ralstonia solanacearum strains and a bacterial wilt disease control method using the bacteriophage.

### Solution to Problem

As a result of extensive studies to solve the problem, the present inventors have found that a bacteriophage in which the amino acid sequence at the C-terminus of a tail fiber protein including a host recognition site is a specific sequence infects a wide variety of Ralstonia solanacearum and thus have completed the invention.

That is, the invention is a bacteriophage characterized in that the amino acid sequence at the C-terminus of a tail fiber protein including a host recognition site is the amino acid sequence of SEQ ID NO: 1 and that the bacteriophage infects Ralstonia solanacearum.

Moreover, the invention is a bacterial wilt disease control agent characterized by containing the bacteriophage as an active component.

Furthermore, the invention is a bacterial wilt disease control method in a plant characterized by administering the bacterial wilt disease control agent to a plant or a plant growth medium.

Further, the invention is the amino acid sequence at the C-terminus of a tail fiber protein including a host recognition site shown in SEQ ID NO: 1 and a base sequence encoding the amino acid sequence.

### Advantageous Effects of Invention

The bacteriophage of the invention infects a wide variety of Ralstonia solanacearum strains and thus is effective for controlling bacterial wilt.

Moreover, by incorporating (or substituting with) the amino acid sequence at the C-terminus of a tail fiber protein including a host recognition site of the invention (SEQ ID NO: 1) into a host recognition site of a tail fiber protein of another bacteriophage or the like by a general method using the amino acid sequence or a base sequence encoding the amino acid sequence, infection of a wide variety of Ralstonia solanacearum strains is enabled.

### BRIEF DESCRIPTION OF DRAWINGS

FIG. 1 shows an example of the appearance of bacteriophage RKP181 of the invention. The length of the bar in the figure is 20 nm.

FIG. 2 shows a phylogenetic tree (Maximum Likelihood) based on the amino acid sequence of the DNA packaging protein B gene of RKP181.

FIG. 3 shows comparison of the maps of major genes of RKP181 and RSB2.

FIG. 4 shows comparison of the whole genome sequences of RKP181 and RSB2.

FIG. 5 shows a phylogenetic tree (Maximum Likelihood) based on the amino acid sequence encoded by the tail fiber gene of RKP181,

FIG. 6 shows comparison of the identities of the tail fiber gene products of RKP181, T7 and RSB2.

FIG. 7 shows one infection cycle of RKP181 (mean±SD (N=3)).

FIG. 8 shows the genetic stability of RKP181 after passage amplification (the left is the full genome length, and the right is after cleavage with the restriction enzyme Eco81I).

### DESCRIPTION OF EMBODIMENTS

The amino acid sequence at the C-terminus of a tail fiber protein including a host recognition site of the bacteriophage of the invention is the amino acid sequence of SEQ ID NO: 1, and the bacteriophage infects Ralstonia solanacearum.

The amino acid sequence at the C-terminus of a tail fiber protein including a host recognition site of the bacteriophage shown in SEQ ID NO: 1 is largely different from the amino acid sequences at the C-terminus of a tail fiber protein including a host recognition site of conventionally known bacteriophages, and as a result, the bacteriophage infects a wide variety of Ralstonia solanacearum strains. For example, the identities of the amino acid sequences at the C-terminus of a tail fiber protein including a host recognition site of RSJ5 and RSB2, which are related bacteriophages of the bacteriophage of the invention, to the amino acid sequence at the C-terminus of a tail fiber protein including a host recognition site of the bacteriophage of the invention are only 38% and 23% according to ClustalW analysis (see the Examples).

As described above, the bacteriophage of the invention infects a wide variety of Ralstonia solanacearum strains because the amino acid sequence at the C-terminus of a tail fiber protein including a host recognition site is the amino acid sequence of SEQ ID NO: 1.

Here, by incorporating (or substituting with) the amino acid sequence at the C-terminus of a tail fiber protein including a host recognition site of the invention (SEQ ID NO: 1) at a host recognition site of a tail fiber protein of another bacteriophage or the like by a general method using the amino acid sequence or a base sequence encoding the amino acid sequence, infection of a wide variety of Ralstonia solanacearum strains is enabled (Document 1. (Ando H, Lemire S, Pires D P, Lu T K. 2015. Engineering Modular Viral Scaffolds for Targeted Bacterial Population Editing. Cell Systems 1:187-196.)). The base sequence encoding the amino acid sequence at the C-terminus of a tail fiber protein including a host recognition site (SEQ ID NO: 1) which the bacteriophage of the invention has is not particularly limited as long as the base sequence corresponds to the amino acid sequence but is preferably the sequence from the 34, 313th to the 35, 968th residues of the base sequence of SEQ ID NO: 3 (the whole genome sequence of RKP181)

The Ralstonia solanacearum infected with the bacteriophage of the invention is not particularly limited as long as the Ralstonia solanacearum is any of those found from 200 plant species or more such as the Solanaceae plants, the Zingiberaceae plants and the plants, the Lamiaceae but bacteriophage infects at least all of strain MAFF107624, strain MAFF211266, strain MAFF211270, strain MAFF211543, strain MAFF301859, strain MAFF311644, strain MAFF730103, strain MAFF730131, strain MAFF302745, strain MAFF311632, strain MAFF211536, strain MAFF331041, strain MAFF730139, strain MAFF211280, strain MAFF211533, strain MAFF211468, strain MAFF211516, strain MAFF311101, strain MAFF311102, strain MAFF211479, strain MAFF211471, strain MAFF211483, strain MAFF211484, strain MAFF211486, strain MAFF211272, strain MAFF211276, strain MAFF211278, strain MAFF211490, strain MAFF211492, strain MAFF211497, strain MAFF211476, strain MAFF211414, MAFF211429 strain strain and MAFF301558 and further infects the 70 Ralstonia solanacearum strains or more collected by the present inventors. In this regard, the Ralstonia solanacearum strains having a number with MAFF can be obtained from Genebank Project, NARO, Agriculture National and Food Research Organization.

In this regard, the infection here refers to the state in which plaques are formed on an agar plate on which Ralstonia solanacearum grows or the state of lysing in a liquid medium in the presence of RKP181.

The bacteriophage of the invention described above can be obtained by screening known bacteriophages according to a general method to find those in which the amino acid sequence at the C-terminus of a tail fiber protein including a host recognition site is the amino acid sequence of SEQ ID NO: 1.

Moreover, the bacteriophage of the invention should have the above amino acid sequence. Thus, the bacteriophage of the invention may belong to any group, any order or any family but preferably belongs to the family Podoviridae of the order Caudovirales of the group I.

The bacteriophage of the invention has a head diameter of 30 to 90 nm, preferably 55 to 66 nm, a tail length of 5 to 30 nm, preferably 11 to 17 nm and a width of 5 to 20 nm, preferably 10 to 17 nm.

The bacteriophage of the invention has a double-stranded genome.

The genome size of the bacteriophage of the invention is not particularly limited but is for example 6,000 to 280, 000 bp, preferably 38, 000 to 40, 000 bp. The genome size is the value of the whole genome base sequence.

The GC content of the bacteriophage of the invention is not particularly limited but is for example 55 to 75%, preferably 61 to 64%. The GC content is the value calculated from the whole genome base sequence,

The number of genes in the bacteriophage of the invention is not particularly limited but is for example 10 to 330 genes, preferably 50 genes. Of the 50 genes, 49 genes are in the same direction.

The genomic DNA of the bacteriophage of the invention is preferably digested into fragments through treatment with the restriction enzyme Eco81I, particularly preferably into five fragments of 2 kb or more. The conditions for treatment with the restriction enzyme Eco81I are at 35 to 40° C. for one to three hours.

Furthermore, the bacteriophage of the invention may have, for example, genes such as lysozyme and typeII holin or may have a circular or linear genome,

A preferable example of the bacteriophage of the invention explained above is RKP181 found by the present inventors. RKP181 was deposited for an international deposit at NITE Patent Microorganisms Depositary, National Institute of Technology and Evaluation (address: #122, 2-5-8 Kazusakamatari, Kisarazu-shi, Chiba 292-0818, Japan) on Mar. 27, 2020 under NITE BP-03186.

The bacteriophage of the invention also includes those having modification, mutation or the like while maintaining the properties.

Because the bacteriophage of the invention can infect a wide variety of Ralstonia solanacearum, a bacterial wilt disease control agent can be obtained using the bacteriophage as an active component.

In this regard, the control here refers to suppression and alleviation of infection of a plant with Ralstonia solanacearum, suppression and alleviation of the onset of bacterial wilt by Ralstonia solanacearum, suppression and alleviation of spread of infection with bacterial wilt disease or control of Ralstonia solanacearum,

The amount of the bacteriophage of the invention in the bacterial wilt disease control agent may be appropriately set depending on the purpose of control, but for example, the bacteriophage of the invention may be contained at potency of 103 to 1012 pfu/mb, preferably 105 to 1011 pfu/mL.

The bacterial wilt disease control agent may contain only the bacteriophage of the invention, which is the active component, but may further contain another substance, a composition of the like such as an agronomically, pharmacologically and botanically acceptable protein stabilizer.

The bacterial wilt disease control agent described above can control bacterial wilt disease in a plant when administered to a plant or a plant growth medium. Examples of the plant include tomatoes, eggplants, bell peppers and tobaccos of the family Solanaceae, Perilla frutescens var. crispa and Perilla frutescens of the family Lamiaceae, ginger, turmeric and curcuma of the family Zingiberaceae and the like. The plant growth medium may be soil, a solid medium such as a mat containing organic materials, a liquid containing a nutrient solution or the like. The method and the conditions for administering the bacterial wilt disease control agent to such a plant or a plant growth medium are not particularly limited but are, for example, spraying or dropping to the plant or the plant growth medium, injection into the plant or the like.

### EXAMPLES

The invention is explained in detail below referring to Examples, but the invention is not limited to the Examples.

### Example 1

### Separation of Bacteriophage:

Soil was suspended in water and then left still, and thus a supernatant was obtained. The supernatant was filtered with a filter of 0.25 μm, and the filtrate was subjected to a plaque assay using Ralstonia solanacearum as the host. By isolating a formed plaque, bacteriophage RKP181 was obtained.

### Example 2

### Analysis of Bacteriophage RKP181:

### (1) Electron Microscope Analysis

A phage suspension of 2×10¹¹ pfu/ml was stained with 1% uranyl acetate, and pictures were taken under an electron (JEM-1400Pus, manufactured by JEOL Ltd.).

### (2) Determination of Genome Size

The genomic DNA was prepared from the phage particles by phenol/chloroform extraction. To determine the size of the genome, the purified genomic DNA was subjected to agarose gel electrophoresis with Mupid-2plus electrophoresis apparatus (manufactured by Mupid) using 0.3% agarose (Agarose H, manufactured by Nippon Gene Co., Ltd.). Moreover, the genomic DNA which was treated with restriction enzymes EcoRI the (manufactured by Toyobo Co., Ltd.) and Eco81I (manufactured by Takara Bio Inc.), respectively was subjected to agarose gel electrophoresis with Mupid-2plus electrophoresis apparatus using 0.8% agarose (Agarose 1200 Standard Type, manufactured by PH Japan Co., Ltd) or 0.3% agarose (Agarose H, manufactured by Nippon Gene Co., Ltd.).

According to the bands of the size marker, the genome size of RKP181 was estimated to be 38,400 to 48,500 bp. Moreover, as a result of comparison with the bands of the size marker, the band sizes obtained by the treatment with EcoRI were 21.1 kb, 16.1 kb, 1.3 kb and 1 kb, and the band sizes obtained by the treatment with Eco81I were 12.9 kb, 10.2 kb, 7 kb, 6.3 kb and 2 kb, which means that there were five fragments of 2 kb or more. It was thus found that the genome size of RKP181 is 38.4 to 39.5 kbp.

### (3) Determination of Genomic Structure

The purified genomic DNA was treated with a single-stranded DNA nuclease (S1 Nuclease, manufactured by Promega Corporation), a DNA nuclease (Recombinant DNase I, manufactured by Takara Bio Inc.), an RNA nuclease (RNase A, manufactured by Takara Bio Inc.) and a linear DNA nuclease (BAL31 nuclease, manufactured by New England Biolabs, Inc.) and subjected to electrophoresis by the method described in the above Example. Moreover, the terminal structures of the genome were determined by treating the genomic DNA which was treated with the linear DNA nuclease (BAL31 nuclease) for different time periods with Eco81I (Takara Bio Inc.) and conducting electrophoresis by the method described in the above Example.

Because the genomic DNA of RKP181 was not digested with the single-stranded DNA nuclease (S1 Nuclease) and the RNA nuclease (RNase A) but was digested with the DNA nuclease (Recombinant DNase I) and the linear DNA nuclease (BAL31 nuclease), it was found that the genome of RKP181 is linear double-stranded DNA. Moreover, the fragments of 12.9 kb, 10.2 kb, 7 kb, 6.3 kb and 2 kb obtained by the treatment with Eco81I were lost with time with the linear DNA nuclease (BAL31 nuclease) from the fragments of 10.2 kb and 6.3 kb,

### (4) Genomic Analysis

The whole genome base sequence of RKP181 was determined with PacBio RS II (manufactured by PACIFIC BIOSCIENCES), The determined base sequences were assembled using RS_HGAP Assembly 2.3.0. To estimate the terminal structures and the lengths of the terminal sequences of the genome, PhageTerm (Galaxy) was used. The terminal redundant sequences were determined by Sanger sequencing (Applied Biosystems 3730xl DNA analyzer). Related strains based on the whole genome sequences were searched by homology search with BLASTN (Table 1). The open reading frames (ORFs) were estimated using MetaGeneAnnotator 1.0 of MiGAP ver2.23 (Database Center for Life Science), tRNAscan-SE 1.23 and BLAST 2.2.18 (Table 2). The functions of the detected ORFs were estimated using the COG, Refseq, TrEMBL and nr databases for those at the top obtained by homology search with MiGAP/BLAST and PSI-BLAST (Table 2). The molecular phylogenetic tree based on the amino acid sequences of the DNA packaging protein B gene was created by the maximum likelihood method by obtaining the amino acid sequences of the DNA packaging protein B gene of the related strains from the NCBI database and aligning the sequences with ClustalW of MEGA 7.0.26 (FIG. 2).

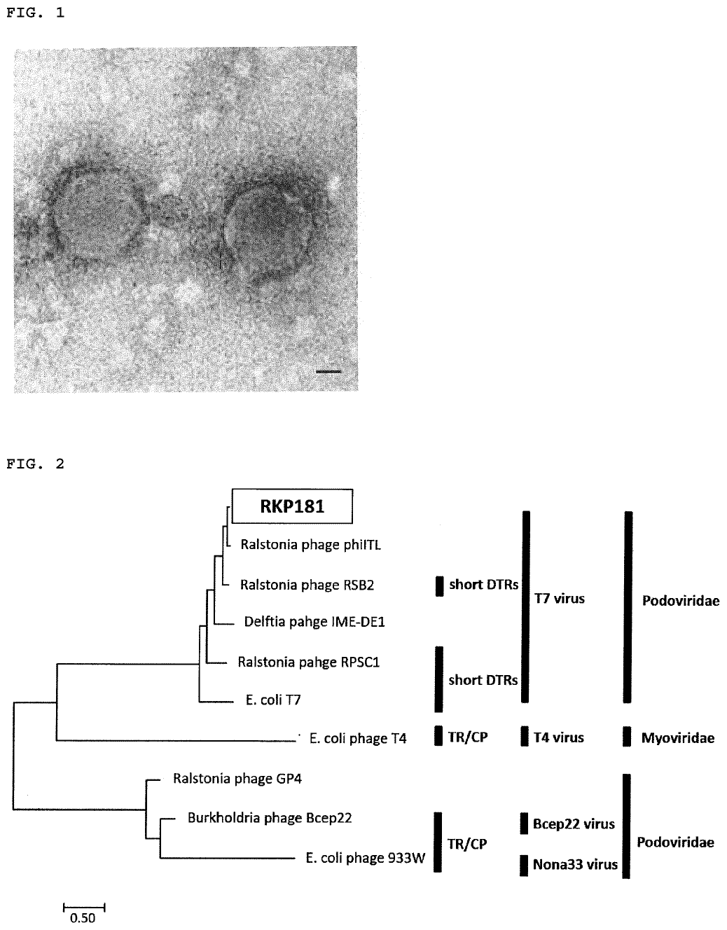

The genome had a size of 39, 455 bp and contained redundant sequences at both ends, and the terminal redundant sequences were 239 bp in length. The GC content of the genome was 62.6%. From the identity comparison of the genomes, RKP181 was related to phages of the genus 7-like virus of the family Podoviridae (podovirus) of the order Caudovirales, which is consistent with the results of the electron microscope analysis (Table 1 and FIGS. 1 and 2). The closest related strain was known Ralstonia phage strain phiITL-1 (Table 1 and FIG. 2), and the identity was 87% (coverage of 84%) according to BLASTN. The next homologous strain was Ralstonia phage strain RSB2 (coverage of 70% and identity of 77%). From the results of the analysis with PhageTerm and the phylogenetic systematic analysis based on the DNA packaging DNA protein B gene (FIG. 2), the terminal structures of the genome of RKP181 were estimated to have a short direct terminal redundancy [short DTR] structure and to be T7 type as described above. The whole genome of RKP181 determined with PacBio RS II is shown in SEQ ID NO: 3. Moreover, as shown in Table 2, the genome of RKP181 included 50 genes in total, of which 49 genes were in the same direction.

TABLE 1

| | | | | | | | | Base | GC | Gene |
|---|---|---|---|---|---|---|---|---|---|---|
| Top | Accession | Species | Coverage | Identity | Family | Genus | Structure | Length (b) | (%) | Number |
| 1 | KP343639.1 | Ralstonia | 84% | 87% | Podoviridae | T7 virus | Circular | 38,731 | 62.8% | 54 |
| | | phage philTL-1 | | | | | | | | |
| 2 | AB597179.1 | Ralstonia | 70% | 77% | Podoviridae | T7 virus | Linear | 40,411 | 61.8% | 53 |
| | | phage RSB2 | | | | | | | | |
| 3 | KR153873.1 | Delftia | 6% | 75% | Podoviridae | T7 virus | Circular | 38,084 | 60.4% | 48 |
| | | phage IME-DE1 | | | | | | | | |
| 4 | MF893341.1 | Ralstonia | 3% | 74% | Podoviridae | T7 virus | Circular | 39628 | 61.6% | 43 |
| | | phage RPSC1 | | | | | | | | |

TABLE 2

| | | | | | | | | | | | homolog |
|---|---|---|---|---|---|---|---|---|---|---|---|
| | Start | End | Length | Length | | | | | | | accession |
| ORF | (nt) | (nt) | (nt) | (aa) | strand | putative function | organism | product | E-value | Ident. | no. |
| DTR | 1 | 239 | 239 | NA | + | direct terminal | NA | NA | NA | NA | NA |
| | | | | | | repeat | | | | | |
| 1 | 404 | 634 | 231 | 76 | − | Signal transduction | Ralstonia | hypothetical | 3E−39 | 84% | AJT60839.1 |
| | | | | | | histidine kinase | phage | protein | | | |
| | | | | | | | philTL-1 | | | | |
| 2 | 1,304 | 1,504 | 201 | 56 | + | NA | Ralstonia | hypothetical | 4E−30 | 76% | AJT60799.1 |
| | | | | | | | phage | protein | | | |
| | | | | | | | philTL-1 | | | | |
| 3 | 1,769 | 2,011 | 243 | 80 | + | NA | Ralstonia | hypothetical | 2E−34 | 70% | AJT60800.1 |
| | | | | | | | phage | protein | | | |
| | | | | | | | philTL-1 | | | | |
| 4 | 2,022 | 2,441 | 420 | 139 | + | Cell division | Ralstonia | hypothetical | 5E−58 | 63% | YP_009017724.1 |
| | | | | | | protein Ftsl/ | phage | protein ORF3 | | | |
| | | | | | | penicillin- | RSB2 | | | | |
| | | | | | | binding protein 2 | | | | | |
| 5 | 2,519 | 2,764 | 246 | 81 | + | Non-ribosomal | Ralstonia | hypothetical | 5E−35 | 70% | YP_009017725.1 |
| | | | | | | peptide synthetase | phage | protein ORF4 | | | |
| | | | | | | modules and | RSB2 | | | | |
| | | | | | | related proteins | | | | | |
| 6 | 2,820 | 3,002 | 183 | 60 | + | NA | Ralstonia | hypothetical | 1E−14 | 52% | YP_009017726.1 |
| | | | | | | | phage | protein ORF5 | | | |
| | | | | | | | RSB2 | | | | |
| 7 | 2,999 | 3,178 | 180 | 59 | + | NA | Ralstonia | hypothetical | 8E−16 | 65% | YP_009017727.1 |
| | | | | | | | phage | protein ORF6 | | | |
| | | | | | | | RSB2 | | | | |
| 8 | 3,175 | 3,495 | 321 | 106 | + | Uncharacterized | Ralstonia | hypothetical | 2E−26 | 59% | YP_009017728.1 |
| | | | | | | bacitracin | phage | protein ORF7 | | | |
| | | | | | | resistance protein | RSB2 | | | | |
| 9 | 3,492 | 3,857 | 366 | 121 | + | Phosphoenol- | Ralstonia | hypothetical | 1E−49 | 72% | YP_009017729.1 |
| | | | | | | pyruvate | phage | protein ORF8 | | | |
| | | | | | | carboxykinase | RSB2 | | | | |
| | | | | | | (GTP) | | | | | |
| 10 | 3,870 | 4,049 | 180 | 59 | + | NA | Ralstonia | hypothetical | 6E−06 | 48% | AJT60804.1 |
| | | | | | | | phage | protein | | | |
| | | | | | | | philTL-1 | | | | |
| 11 | 4,075 | 4,341 | 267 | 88 | + | NA | Ralstonia | hypothetical | 1E−37 | 73% | AJT60805.1 |
| | | | | | | | phage | protein | | | |
| | | | | | | | philTL-1 | | | | |
| 12 | 4,320 | 4,586 | 267 | 88 | + | Ribonucleases | Ralstonia | hypothetical | 1E−39 | 78% | AJT60806.1 |
| | | | | | | G and E | phage | protein | | | |
| | | | | | | | philTL-1 | | | | |
| 13 | 4,651 | 5,007 | 357 | 118 | + | NA | Ralstonia | hypothetical | 4E−46 | 85% | AJT60808.1 |
| | | | | | | | phage | protein | | | |
| | | | | | | | philTL-1 | | | | |
| 14 | 5,058 | 5,466 | 408 | 135 | + | Enoyl reductase | Ralstonia | hypothetical | 1E−42 | 77% | AJT60810.1 |
| | | | | | | domain of yeast- | phage | protein | | | |
| | | | | | | type FAS1 | philTL-1 | | | | |
| 15 | 5,489 | 5,570 | 102 | 33 | + | NA | Ralstonia | hypothetical | 1E−12 | 91% | YP_009017736.1 |
| | | | | | | | phage | protein | | | |
| | | | | | | | RSB2 | | | | |
| 16 | 5,567 | 6,499 | 933 | 310 | + | ATP-dependent | Ralstonia | ATP-dependent | 7E−110 | 54% | AJT60812.1 |
| | | | | | | DNA ligase | phage | DNA ligase | | | |
| | | | | | | | philTL-1 | | | | |
| 17 | 6,496 | 6,615 | 120 | 39 | + | NA | Spirosoma | M56 family | 0.5 | 49% | WP_020605153.1 |
| | | | | | | | spitsbergense | metallo- | | | |
| | | | | | | | | peptidase | | | |
| 18 | 6,768 | 9,365 | 2,598 | 865 | + | Mitochondrial | Ralstonia | DNA-directed | 0E+00 | 80% | YP_009017739.1 |
| | | | | | | DNA-directed RNA | phage | RNA | | | |
| | | | | | | polymerase | RSB2 | polymerase | | | |
| 19 | 9,385 | 9,831 | 447 | 148 | + | NA | Ralstonia | hypothetical | 4E−57 | 59% | AJT60815.1 |
| | | | | | | | phage | protein | | | |
| | | | | | | | philTL-1 | | | | |
| 20 | 10,012 | 10,257 | 246 | 81 | + | NA | Ralstonia | hypothetical | 9E−05 | 53% | AJT60816.1 |
| | | | | | | | phage | protein | | | |
| | | | | | | | philTL-1 | | | | |
| 21 | 10,254 | 10,553 | 300 | 99 | + | Flp pilus | Ralstonia | hypothetical | 5E−24 | 62% | AJT60817.1 |
| | | | | | | assembly | phage | protein | | | |
| | | | | | | protein TadC | philTL-1 | | | | |
| 22 | 10,616 | 10,921 | 308 | 101 | + | NA | Ralstonia | hypothetical | 2E−60 | 89% | AJT60818.1 |
| | | | | | | | phage | protein | | | |
| | | | | | | | philTL-1 | | | | |
| 23 | 10,914 | 11,609 | 696 | 231 | + | AraC-type | Ralstonia | hypothetical | 6E−93 | 68% | YP_009017744.1 |
| | | | | | | DNA-binding | phage | protein | | | |
| | | | | | | domain-containing | RSB2 | ORF23 | | | |
| | | | | | | proteins | | | | | |
| 24 | 11,590 | 11,763 | 174 | 57 | + | deoxynucleoside | Ralstonia | hypothetical | 7E−17 | 67% | AJT60821.1 |
| | | | | | | monophosphate | phage | protein | | | |
| | | | | | | kinase | philTL-1 | | | | |
| 25 | 11,831 | 12,496 | 666 | 22 | + | NA | Ralstonia | Putative | 1E−138 | 91% | AIT60822.1 |
| | | | | | | | phage | ssDNA binding | | | |
| | | | | | | | philTL-1 | protein | | | |
| 26 | 12,499 | 12,966 | 468 | 155 | + | single-stranded | Ralstonia | T7-like phage | 2E−105 | 94% | AJT60823.1 |
| | | | | | | DNA-binding | phage | endonuclease | | | |
| | | | | | | protein | philTL-1 | | | | |
| 27 | 12,968 | 13,438 | 471 | 156 | + | Formamido- | Ralstonia | lysozyme | 5E−61 | 63% | YP_009017748.1 |
| | | | | | | pyrimidine- | phage | [Peplidoglycan | | | |
| | | | | | | DNA glycosylase | RSB2 | recognition | | | |
| | | | | | | | | proteins] | | | |
| 28 | 13,545 | 15,230 | 1,586 | 561 | + | lysozyme(N- | Ralstonia | DNA primase/ | 0E+00 | 90% | AJT60826.1 |
| | | | | | | acetylmuramoyl-L- | phage | helicase-like | | | |
| | | | | | | alanine amidase) | philTL-1 | protein | | | |
| 29 | 15,240 | 17,348 | 2,109 | 702 | + | Replicative | Ralstonia | DNA | 0E+00 | 95% | AJT60827.1 |
| | | | | | | DNA helicase | phage | polymerase″ | | | |
| | | | | | | | philTL-1 | | | | |
| 30 | 17,398 | 17,775 | 378 | 125 | + | DNA polymerase | Ralstonia | hypothetical | 8E−56 | 78% | AJT60828.1 |
| | | | | | | I - 3′-5′ | phage | protein | | | |
| | | | | | | exonuclease and | philTL-1 | | | | |
| | | | | | | polymerase | | | | | |
| | | | | | | domains | | | | | |
| 31 | 17,798 | 17,998 | 201 | 56 | + | NA | Pectobacterium | HNS binding | 2E−18 | 55% | ATN92966.1 |
| | | | | | | | phage | protein | | | |
| | | | | | | | PPWS4 | | | | |
| 32 | 17,995 | 18,879 | 885 | 294 | + | Glycosyltransferase | Ralstonia | phage | 0E+00 | 95% | AJT60830.1 |
| | | | | | | | phage | exonuclease | | | |
| | | | | | | | philTL-1 | | | | |
| 33 | 18,901 | 19,212 | 312 | 103 | + | 5′-3′ exonuclease | Ralstonia | hypothetical | 7E−36 | 67% | AJT60831.1 |
| | | | | | | (including | phage | protein | | | |
| | | | | | | N-terminal | philTL-1 | | | | |
| | | | | | | domain of Poll) | | | | | |
| 34 | 19,226 | 19,591 | 366 | 121 | + | Fe2+ transport | Ralstonia | hypothetical | 3E−63 | 76% | AJT60832.1 |
| | | | | | | system protein B | phage | protein | | | |
| | | | | | | | philTL-1 | | | | |
| 35 | 19,594 | 19,896 | 303 | 100 | + | Uncharacterized | Ralstonia | hypothetical | 2E−27 | 82% | AJT60833.1 |
| | | | | | | conserved protein | phage | protein | | | |
| | | | | | | | philTL-1 | | | | |
| 36 | 19,901 | 21,496 | 1,596 | 531 | + | Small-conductance | Ralstonia | Putative head | 0E+00 | 92% | AJT60834.1 |
| | | | | | | mechanosensitive | phage | to tail joining | | | |
| | | | | | | channel | philTL-1 | protein (collar) | | | |
| 37 | 21,557 | 22,381 | 825 | 274 | + | Transcriptional | Ralstonia | Putative | 1E+00 | 84% | AJT60835.1 |
| | | | | | | regulators | phage | capsid | | | |
| | | | | | | | philTL-1 | assembly | | | |
| | | | | | | | | protein | | | |
| 38 | 22,494 | 23,453 | 960 | 319 | + | Phage T7 capsid | Ralstonia | Putative | 0E+00 | 93% | AJT60836.1 |
| | | | | | | assembly protein | phage | major capsid | | | |
| | | | | | | | philTL-1 | protein | | | |
| 39 | 23,560 | 24,138 | 579 | 192 | + | major capsid | Ralstonia | Putative tail | 8E−123 | 87% | AJT60837.1 |
| | | | | | | protein | phage | tubular | | | |
| | | | | | | | philTL-1 | protein A | | | |
| 40 | 24,141 | 25,507 | 2,367 | 788 | + | T7 tail tubular | Ralstonia | Putative tail | 0E+00 | 80% | YP_009017762.1 |
| | | | | | | gp11 protein | phage | tubular | | | |
| | | | | | | | RSB2 | protein B | | | |
| 41 | 26,615 | 27,079 | 465 | 154 | + | FOG: WD40 repeat | Ralstonia | Putative | 2E−104 | 94% | AJT60786.1 |
| | | | | | | | phage | internal | | | |
| | | | | | | | philTL-1 | virion | | | |
| | | | | | | | | protein A | | | |
| 42 | 27,072 | 27,653 | 582 | 193 | + | Putative internal | Ralstonia | Putative | 2E−108 | 87% | AJT60787.1 |
| | | | | | | virion protein A | phage | internal | | | |
| | | | | | | | philTL-1 | virion | | | |
| | | | | | | | | protein B | | | |
| 43 | 27,663 | 29,846 | 2,184 | 727 | + | Putative internal | Ralstonia | Putative | 0E+00 | 83% | AJT60788.1 |
| | | | | | | virion protein B | phage | internal | | | |
| | | | | | | | philTL-1 | virion | | | |
| | | | | | | | | protein C | | | |
| 44 | 29,865 | 33,749 | 3,885 | 1,294 | + | Putative internal | Ralstonia | Putative | 0E+00 | 88% | AJT60789.1 |
| | | | | | | virion protein C | phage | internal | | | |
| | | | | | | | philTL-1 | virion | | | |
| | | | | | | | | protein D | | | |
| 45 | 33,833 | 35,958 | 2,136 | 711 | + | Soluble lytic | Ralstonia | putative | 4E−107 | 42% | YP_009218128.1 |
| | | | | | | murein | phage RSJ5 | phage | | | |
| | | | | | | transglycosylase | | tail fiber | | | |
| | | | | | | and related | | protein | | | |
| | | | | | | regulatory proteins | | | | | |
| | | | | | | (some contain | | | | | |
| | | | | | | LysM/invasin | | | | | |
| | | | | | | domains) | | | | | |
| 46 | 35,968 | 36,294 | 327 | 108 | + | Phage T7 tail | Ralstonia | Putative | 4E−68 | 96% | AJT60793.1 |
| | | | | | | fibre protein | phage | uncharacterized | | | |
| | | | | | | | philTL-1 | protein | | | |
| 47 | 36,294 | 36,506 | 213 | 70 | + | AraC-type DNA- | Ralstonia | Putative | 4E−35 | 84% | AJT60794.1 |
| | | | | | | binding domain- | phage | lysis protein | | | |
| | | | | | | containing proteins | philTL-1 | | | | |
| 48 | 36,503 | 36,775 | 273 | 90 | + | type II holin | Ralstonia | Putative | 4E−35 | 84% | AJT60794.1 |
| | | | | | | | phage | packaging | | | |
| | | | | | | | philTL-1 | maturation | | | |
| | | | | | | | | protein A | | | |
| 49 | 36,798 | 38,579 | 1,782 | 593 | + | DNA packaging | Ralstonia | Putative | 0E+00 | 94% | AJT60797.1 |
| | | | | | | protein, small | phage | packaging | | | |
| | | | | | | subunit | philTL-1 | maturation | | | |
| | | | | | | | | protein B | | | |
| 50 | 38,795 | 39,007 | 213 | 70 | + | NA | Ralstonia | hypothetical | 5E−20 | 77% | AJT60798.1 |
| | | | | | | | phage | protein | | | |
| | | | | | | | philTL-1 | | | | |
| DTR | 39,217 | 39,455 | 239 | NA | + | Direct terminal | NA | NA | NA | NA | NA |
| | | | | | | repeat | | | | | |

### (5) Genetic Map

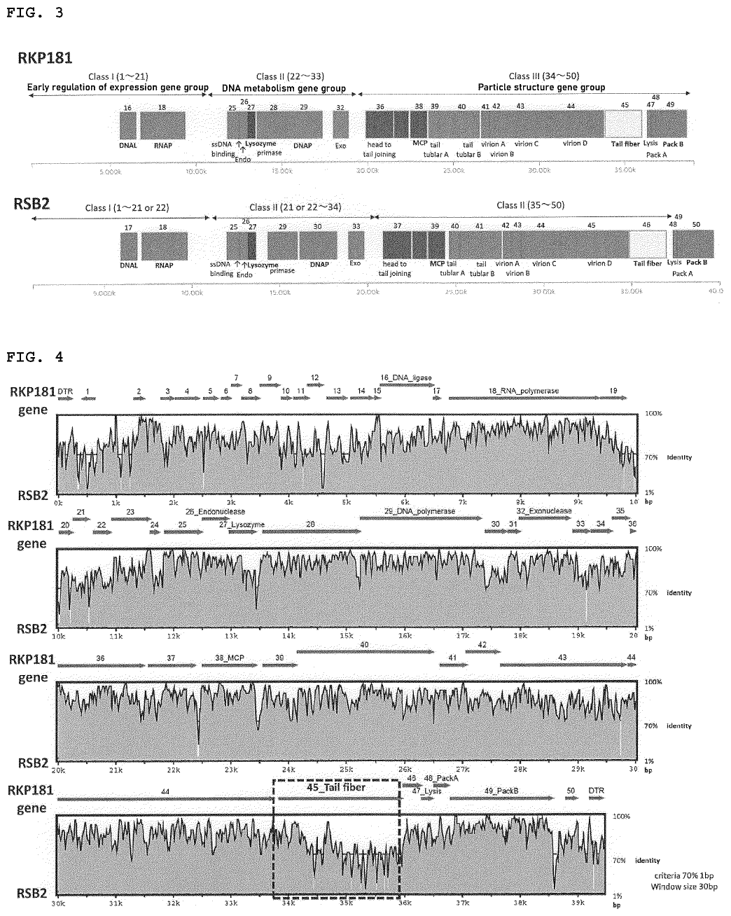

Maps of major genes in the genomes were drawn using PHASTER, and RKP181 and Ralstonia phage RSB2 (AB597179.1) of JP-A-2018-24589 were compared (FIG. 3). Moreover, the entire base sequences were also compared using comparative genomic software LAGAN (FIG. 4).

As in RSB2, the genome of RKP181 included three distinct function modules of class I, class II and class III (FIG. 3, Document 2 (Kawasaki T, Narulita E, Matsunami M, Ishikawa H, Shimizu M, Fujie M, Bhunchoth A, Phironrit N, Chatchawankanphanich O, Yamada T. 2016. Genomic diversity of large-plaque-forming podoviruses infecting the phytopathogen Ralstonia solanacearum. 492:73-81.) Virology and Document 3 (JP-A-2018-24589)). Moreover, because the T-type RNA polymerase gene (RNAP) is in class I in the genome of RKP181 as in RSB2, it is believed that the infection cycle period is shorter than those of other bacteriophages due to rapid expression of RNA polymerase like RSB2 (FIG. 3, see below).

Although the whole genome base sequence and the genetic map of RKP181 are similar to those of RSB2, regarding the tail fiber gene, the identity of ORF45 of RKP181 and the homolog of RSB2 is significantly low (see below),

### (6) Analysis of Tail Fiber Protein-Encoding Gene (Tail Fiber Gene)

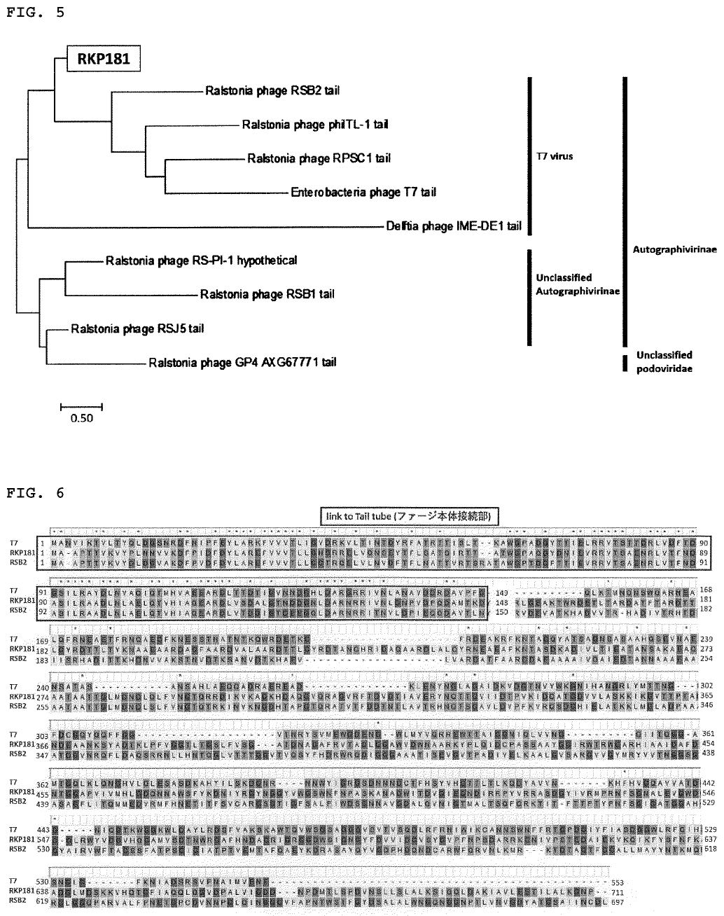

Homology search of the amino acid sequence of ORF45 (SEQ ID NO: 2) encoded by the tail fiber gene of RKP181 was conducted using BLASTP (Table 3). Moreover, the amino acid sequence of ORF45 and known homologous tail fiber gene products were aligned with ClustalW of MEGA 7.0.26, and a molecular phylogenetic tree was created by the maximum likelihood method (FIG. 5). Further, the domains of ORF45 were searched using BLASTP. The identity comparison of the sequence of the 1-149th amino acids of the phage body (tail tube) linker site of the tail fiber gene (AAM43540.1) of T7 and the 1-148th amino acids of ORF45 (shown in SEQ ID NO: 2) of RKP181 was conducted with BLASTP. Subsequently, homology search of the two identified domains, namely, the sequences of the 1-160th amino acids at the N-terminus and the 161-711th amino acids at the C-terminus, was conducted with BLASTP (Table 4 and Table 5). The identities of the tail fiber gene products of RKP181, T7 and RSB2 were compared by obtaining the sequence (BAJ51834.1) of the tail fiber gene (ORF46) of RSB2 and the sequence (P03748.1) of tail fiber gene gp17 of T7 from the NCBI database and comparing the identities using ClustalW of MEGA 7.0.26 (FIG. 6).

As a result of the homology search with BLASTP, the identity of ORF45 of RKP181 to the tail fiber protein of Ralstonia phage RSJ5 was the highest (coverage of 64% and identity of 42%) (Table 3). Moreover, the identity to the tail fiber protein of Ralstonia phage RSB2 was 53% (coverage of 53%), and the identity to the tail fiber protein of Ralstonia phage phiITL-1 was 85% (coverage of 22%). The identity to the tail fiber protein of Enterobacteria phage T7 was 42% (coverage of 37%). Furthermore, when the identities of the full-length tail fiber proteins were compared by the analysis with ClustalW, the identity of ORF45 of RKP181 to the tail fiber protein of RSJ5 was 38%, and that to the tail fiber protein of RSB2 was 23%, which were found to be significantly lower than the identities obtained by the base sequence comparison of the whole genomes (Table 1). The analysis results show that the amino acid sequence of ORF45 (shown in SEQ ID NO: 2) encoded by the tail fiber gene of RKP181 is unique (see below).

TABLE 3

| Top | Protein | Species | Coverage | Identity | Accession |
|---|---|---|---|---|---|
| 1 | putative phage tail fiber protein | Ralstonia phage RSJ5 | 64% | 42% | YP_009218128.1 |
| 2 | putative tail fiber protein | Ralstonia phage RSB2 | 53% | 53% | YP_009017767.1 |
| 3 | putative tail fiber protein | Ralstonia phage philTL-1 | 22% | 85% | AJT60790.1 |
| 4 | tail fibers protein | Escherichia phage 64795_ec1 | 37% | 43% | YP_009291518.1 |
| 5 | tail fiber protein | Escherichia phage C5 | 38% | 43% | AYD80209.1 |
| 6 | hypothetical protein | Ralstonia solanacearum | 67% | 32% | WP_064049197.1 |
| 7 | tail fiber protein | Escherichia phage HZ2R8 | 65% | 44% | AUV62662.1 |
| 8 | tail fiber protein | Pectobacterium phage PP74 | 34% | 44% | APD19655.1 |
| 9 | tail fiber protein | Enterobacteria phage T7 | 37% | 42% | AAM43540.1 |
| 10 | hypothetical protein | Pseudomonas chlororaphis | 20% | 64% | WP_053269386.1 |

As a result of the comparison of the identities based on the whole genome sequences, RKP181 was related to T7-like bacteriophages, RSB2 and phiITL-1 (Table 1), and similar results were obtained from the molecular phylogenetic analysis based on the amino acid sequences of the DNA packaging protein B gene (FIG. 2). When a molecular phylogenetic tree was created using the amino acid sequences of the tail fiber gene, however, RKP181 belonged to a different clade from that including RSB2, phiITL-1 and T7 unlike in FIG. 2 (FIG. 5).

The tail fiber gene of bacteriophage T7 has a phage body (tail tube) linker site at the 1-149th amino acids at the N-terminus and has a host recognition site (tip domain) at the 465-553rd amino acids at the C-terminus (Document 4 (Steven A C, Trus B L, Maizel J V, Unser M, Parry D A D, Wall J S, Hainfeld J F, Studier F W, 1988. Molecular substructure of a viral receptor-recognition protein: The gp17 tail-fiber of bacteriophage T7. J Mol Biol 200:351-365.) and Document 5 (Garcia-Doval C, Raaij M J van. 2012. Structure of the receptor-binding carboxy-terminal domain of bacteriophage T7 tail fibers. PNAS 109:9390-9395.)).

When the domains of ORF45 of RKP181 were searched using BLASTP, the tail fibre protein (pfam03906) of T7 and the chaperone domain (pfam13884) of a phage protein, endosialidase were identified. Regarding the tail fibre protein (pfam03906), an identity of 58% (coverage of 96%) was detected between the sequence of the 1-149th amino acids of the phage body (tail tube) linker site of the tail fiber gene (AAM43540.1) of T7 and the 1-148th amino acids of ORF45 (shown in SEQ ID NO: 2) of RKP181. When BLASTP search was conducted using the sequence of the 1-148th amino acids of ORF45 of RKP181, the identities to the homologous sites of the tail fiber proteins of Ralstonia phage phiITL-1 and Ralstonia phage RSB2 were 85% (coverage of 100%) and 73% (coverage of 100%), respectively. Furthermore, as a result of the analysis using BLASTP and ClustalW, the identities of the 1-160th amino acids of ORF45 of RKP181 were largely different from those of the subsequent sequence at the C-terminus (Tables 4 and 5). In other words, the 1-160th amino acids at the N-terminus of ORF45 of RKP181 had an identity of 85% (coverage of 99%) to the homologous region of the tail fiber protein of phiITL-1, an identity of 72% (coverage of 99%) to that of RSB2 and an identity of 58% (coverage of 89%) to that of T7 (Table 4).

TABLE 4

| Top | Protein | Species | Coverage | Identity | Accession |
|---|---|---|---|---|---|
| 1 | putative tail fiber protein | Ralstonia phage philTL-1 | 99% | 85% | AJT60790.1 |
| 2 | putative tail fiber protein | Ralstonia phage RSB2 | 99% | 72% | YP_009017767.1 |
| 3 | hypothetical protein | Pseudomonas chlororaphis | 92% | 64% | WP_053269386.1 |
| 4 | Phage tail fibers | Yersinia phage fPS-59 | 96% | 57% | SOO46827.1 |
| 5 | putative tail fiber protein | Erwinia phage | 90% | 59% | QEQ94708.1 |
| | | pEp_SNUABM_09 | | | |
| 38 | tail fiber protein | Escherichia phage T7 | 89%% | 58% | AAM43534.1 |

As described above, the N-terminus side of the tail fiber protein, which is the phage body (tail tube) linker site, was widely conserved among known bacteriophages and especially was a domain with a high identity between RKP181 and related bacteriophages. On the other hand, the domain at the C-terminus involving in the host recognition varied among the bacteriophages. The bacteriophages that have a tail fiber gene in which the chaperon domain (pfam13884) of endosialidase is located at the C-terminus and that infect Ralstonia solanacearum, like RKP181, were RSJ5 and RS-PI-1 only. Moreover, when homology search was conducted with BLASTP using the sequence of the 161-711th amino acids of RKP181 excluding the phage body linker site, the identities of the C-terminus side involving in the host recognition were significantly lower than those of the N-terminus side of the phage body (tail tube) linker site, with an identity of 42% (coverage of 83%) even to the most homologous tail fiber protein of RSJ5 and an identity of 35% (coverage of 60%) to the tail fiber protein of RS-PI-1 (Table 5). Similarly, the identity to the tail fiber protein of RSB2, which does not have the chaperon domain (pfam13884), was significantly low, namely 51% (coverage of 17%).

TABLE 5

| Top | Protein | Species | Coverage | Identity | Accession |
|---|---|---|---|---|---|
| 1 | putative phage tail fiber | Ralstonia phage RSJ5 | 83% | 42% | YP_009218128.1 |
| | protein | | | | |
| 2 | hypothetical protein | Ralstonia solanacearum | 64% | 35% | WP_064049197.1 |
| 3 | hypothetical protein | Burkholderia ubonensis | 61% | 37% | KVW40325.1 |
| | WK94_23500 | | | | |
| 4 | hypothetical protein | Burkholderia ubonensis | 59% | 37% | WP_143135316.1 |
| 5 | hypothetical protein | Ralstonia solanacearum | 88% | 31% | WP_049842194.1 |
| 6 | hypothetical protein | Burkholderia ubonensis | 58% | 37% | WP_060368006.1 |
| 7 | hypothetical protein | Burkholderia ubonensis | 58% | 37% | WP_060063309.1 |
| 8 | hypothetical protein | Burkholderia ubonensis | 58% | 37% | WP_059841529.1 |
| 9 | hypothetical protein | Burkholderia ubonensis | 58% | 37% | WP_060032179.1 |
| 10 | hypothetical protein | Burkholderia ubonensis | 58% | 37% | WP_060163429.1 |
| 11 | hypothetical protein | Burkholderia ubonensis | 58% | 37% | WP_059872146.1 |
| 12 | hypothetical protein | Ralstonia phage RS-PI-1 | 60% | 35% | AQT27772.1 |

### (7) Summary

From the above results, it was found that bacteriophage RKP181 has the following properties.
RKP181 is classified to the genus 77-like virus of the family Podoviridae (podovirus) of the order Caudovirales.
The genome of RKP181 is linear double-stranded DNA.
The genomic structure is type 17 having a short direct terminal redundant sequence structure.
The genome size is 39,200 bp to 39,500 bp, preferably 39,455 bp (including two terminal redundant sequences) or 39,216 bp (including one terminal redundant sequence).
The ends of the genome may have a terminal redundant sequence of 200 to 250 bp, preferably 239 bp.
The GC content is 62.6%.
The closest related strain of known strains is Ralstonia phage strain phiITL-1, with an identity of 87%. The next closest strain is Ralstonia phage strain RSB2, with an identity of 77%.
The amino acid sequence at the C-terminus of a tail fiber protein including a host recognition site is the amino acid sequence of SEQ ID NO: 1.

### Example 3

Separation and Control of Ralstonia solanacearum:
(1) Separation from Disease Plant

A stem of a disease plant was cut, and bacterial ooze was collected. The bacterial ooze was appropriately diluted with distilled water. The diluted solution was applied to Modified SMSA medium, and formed colonies was separated onto CPG agar plates (10 g of peptone, 1 g of casamino acid, 5 g of glucose and 1.7% agar per 1 L).

(2) Separation from Soil

Soil and water were mixed well and left still, and then the supernatant was separated. The separated supernatant was appropriately diluted with water, and the diluted solution was applied to Modified SMSA medium. Formed colonies was separated onto CPG agar plates.

### <Modified SMSA Medium (per 1 L)>

| | | |
|---|---|---|
| Peptone | 10 | g |
| Glucose | 5 | g |
| Casamino acid | 1 | g |
| Agar | 18 | g |
| Bacitracin (10 mg/mL) | 2.5 | mL |
| Polymyxin B sulfate (50 mg/mL) | 2 | mL |
| Chloramphenicol (10 mg/mL) | 0.5 | mL |
| Penicillin G potassium (1 mg/mL) | 0.5 | mL |
| Crystal violet (1 mg/mL) | 5 | mL |
| Tetrazolium chloride (10 mg/mL) | 5 | mL |

### (3) Results

One strain of Ralstonia solanacearum was separated from one field by the above method, and 70 Ralstonia solanacearum strains or more in total from the hosts of the family Solanaceae, the family Zingiberaceae, the family Lamiaceae and the like were separated.

### Example 4

### Examination of Host Range of RKP181:

### (1) Presence or Absence of Infection

The presence or the absence of infection was examined by a plaque assay or a spot test.

### (2) Plaque Assay

The Ralstonia solanacearum strains were cultured at 28° C. overnight in CPG medium (10 g of peptone, 1 g of casamino acid and 5 g of glucose per 1 L). The bacterial culture was adjusted to OD₆₀₀ of 0.25 with CPG medium. A serial dilution of a phage solution was prepared. The bacterial solutions and the diluted phage solutions were mixed and left at 28° C. After 30 minutes, the bacterium/phage mixtures were mixed with 3 ml of top agar (3 g of peptone, 0.3 g of casamino acid, 1.7 g of glucose and 5 g of agar per 1 L) and layered onto CPG agar plates. After culturing at 28° C. overnight, the presence or the absence of infection was observed with the presence or the absence of plaques.

### (3) Spot Test

Culture solutions of the Ralstonia solanacearum strains which were cultured at 28° C. overnight in the CPG medium were adjusted to OD₆₀₀ of 0.25 with CPG medium. The bacterial solutions in a volume of 250 μL and 3 ml of top agar were mixed and layered onto CPG agar plates. After top agar was hardened, a phage solution was spotted, and the presence of the absence of plaques was observed to examine the presence or the absence of infection,

### (4) Results

It was found that RKP181 infects all the 100 Ralstonia solanacearum strains or more in total from the hosts of the family Solanaceae, the family Zingiberaceae, the family Lamiaceae and the like separated from the nature, including the 34 strains listed in Table 6, which can be obtained from Genebank Project, NARO, National Agriculture and Food Research Organization (Tsukuba, Ibaraki),

TABLE 6

| Bacterial Strain | Source of Separation |
|---|---|
| MAFF107624 | Tomato |
| MAFF211266 | Tomato |
| MAFF211270 | Tomato |
| MAFF211543 | Tomato |
| MAFF301859 | Tomato |
| MAFF311644 | Tomato |
| MAFF730103 | Tomato |
| MAFF730131 | Tomato |
| MAFF302745 | Tomato |
| MAFF311632 | Tomato |
| MAFF211536 | Tomato |
| MAFF331041 | Tomato |
| MAFF730139 | Eggplant |
| MAFF211280 | Eggplant |
| MAFF211533 | Eggplant |
| MAFF211468 | Chili pepper |
| MAFF211516 | Bell pepper and sweet green pepper |
| MAFF311101 | Bell pepper and sweet green pepper |
| MAFF311102 | Bell pepper and sweet green pepper |
| MAFF211479 | Ginger |
| MAFF211471 | Ginger |
| MAFF211483 | Ginger |
| MAFF211484 | Ginger |
| MAFF211486 | Ginger |
| MAFF211272 | Curcuma |
| MAFF211276 | Curcuma |
| MAFF211278 | Curcuma |
| MAFF211490 | Myoga ginger |
| MAFF211492 | Myoga ginger |
| MAFF211497 | Myoga ginger |
| MAFF211476 | Ginger |
| MAFF211414 | Potato |
| MAFF211429 | Potato |
| MAFF301558 | Potato |

### Example 5

### Evaluation of Infection Cycle by One-Step Growth Method

After mixing 990 μL of a culture solution of Ralstonia solanacearum strain MAFF730131 cultured to OD₆₀₀ of 0.15 in CPG medium and 10 μL of a RKP181 phage solution (2×10¹⁰ pfu/mL), the mixture was left still at room temperature to adsorb the bacterium and the phage. After 10 minutes, the mixture was centrifuged at 5,000×g for 10 minutes, and the supernatant was collected. Then, the plaque assay was conducted, and the number of adsorbed phages was calculated. The precipitates were re-suspended in 1 mL of CPG medium, and 150 μL was then added to 29, 850 ML of CPG medium, followed by culturing with shaking at 28° C. For 100 minutes after starting culturing with shaking, a 10-μL sample was taken every 10 minutes and mixed with 990 AL of CPG medium. After mixing, a serial dilution was prepared according to the need, and 10 μL was taken immediately, added to 250 μL of a culture solution of strain MAFF730131 which was adjusted to OD₆₀₀ of 0.22 to 0.24 and stirred. The total volume thereof was added immediately to top agar, mixed and then layered onto CPG medium. The number of formed plaques was counted, and potency was calculated. From the potency and the adsorbed phage numbers at the each time point, the phage number generated in one infection cycle (burst size) was calculated. The results are shown in FIG. 7 and summarized in Table 7.

TABLE 7

| Latent Period | Rise Period | Infection Cycle | Burst Size |
|---|---|---|---|
| 30 minutes | 50 minutes | 80 minutes | 61 ± 7 pfu |

From the above results, it was found that the latent period of RKP181 is 30 minutes and that the infection cycle is 80 minutes. It was also found that the phage number generated in one infection cycle is 6117 pfu.

### Example 6

### Genetic Stability:

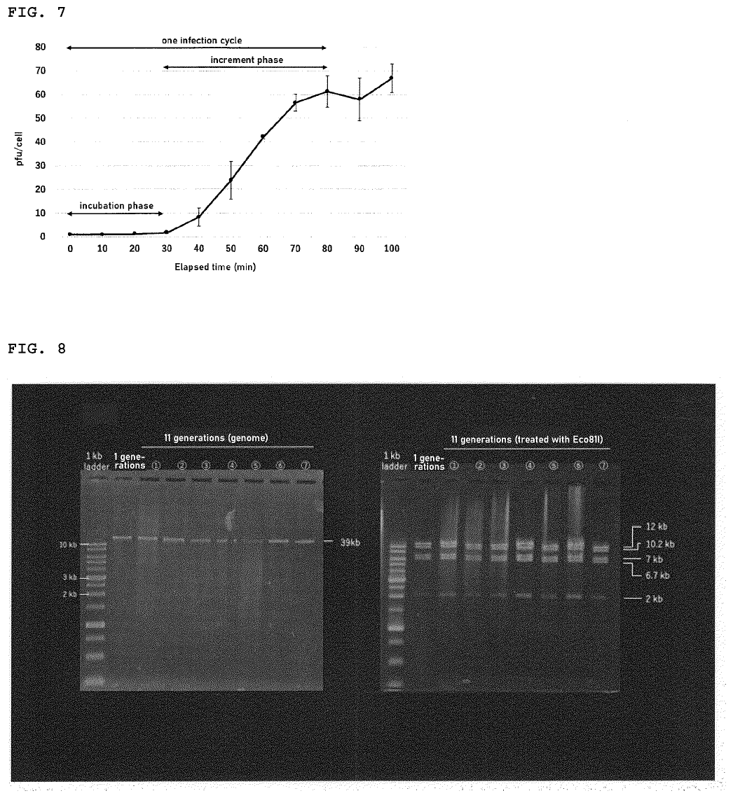

RKP181 was amplified by passaging for 10 generations, and seven plaques were isolated. The genomes were prepared after amplifying the isolated seven phages for one more generation, and the genome sizes were determined by the electrophoresis (FIG. 8: left). Moreover, the prepared genomes were treated with Eco81I restriction enzyme. The restriction patterns were examined and observed to be consistent with the matters described in Example 1 above, and thus RKP181 was found to be genetically stable (FIG. 8: right).

### Example 7

### Control Test:

A 1×10⁹ pfu/mL phage solution in a volume of 5 mL was poured to the roots of seedlings on a cell seedling tray (10 seedlings) of a large tomato variety, Sekaiichi. The control group (10 seedlings) was not treated. After six days, the seedlings were planted in pots, and 5 mL of a bacterial culture solution of strain MAFF730131 which was adjusted to OD₆₆₀=0.1 (corresponding to about 1×10⁸ cfu/mL) was poured to the roots of the seedlings. Then, observation was continued for 22 days. The results are shown in Table 8.

TABLE 8

| | | Day 13 | | Day 20 | | Day 22 | |
|---|---|---|---|---|---|---|---|
| | | | Control | | Control | | Control |
| | | | Value | | Value | | Value |
| Without | Incidence Rate | 40 | — | 50 | — | 60 | — |
| Treatment | Disease Severity | 35 | | 50 | | 60 | |
| RKP181 | Incidence Rate | 20 | 50 | 30 | 40 | 30 | 50 |
| Treatment | Disease Severity | 20 | | 30 | | 30 | |

On the 22nd day after the bacterium inoculation, six of the 10 plants withered in the phage-untreated group, and four plants were healthy. On the other hand, in the RKP181 phage-treated group, withering was limited to three of the 10 plants, and seven plants were healthy, with the control value of 50. Thus, the control property of the RKP181 phage was observed,

### Example 8

### Preparation of Bacterial Wilt DISEASE Control Agent:

Bacteriophage RKP181 separated in Example 1 was adjusted to potency of 1×10⁹ pfu/ml, and a bacterial wilt disease control agent was thus obtained.

### INDUSTRIAL APPLICABILITY

The bacteriophage of the invention can be used for controlling bacterial wilt,

1-8. (canceled)

9. A bacteriophage comprising a tail protein comprising a host recognition site comprising the amino acid sequence of SEQ ID NO: 1.

10. The bacteriophage of claim 9, wherein the host recognition site is produced by inserting an exogenous host recognition site comprising SEQ ID NO: 1.

11. The bacteriophage of claim 9, wherein the host recognition iste is produced by modifying or mutating genomic DNA of a bacteriophage.

12. The bacteriophage of claim 9 that is RKP181 (NITE BP-03186).

13. The bacteriophage of claim 9, which belongs to family Podoviridae of order Caudovirales of Group I.

14. The bacteriophage of claim 13, which has a head diameter in a range of from 30 to 90 nm, a tail length in a range of from 5 to 30 nm, and a width in a range of from 5 to 20 nm, has a double-stranded genome, has a genome size in a range of from 6,000 to 280,000 bp, has a GC content in a range of from 55 to 75% and comprises from 10 to 330 genes, wherein genomic DNA of the bacteriophage is digested into fragments by restriction enzyme Eco81I.

15. The bacteriophage of claim 9 that further comprises a gene encoding lysozyme or type II holin.

16. The bacteriophage of claim 9 that further comprises a circular or linear genome.

17. The bacteriophage of claim 9 that infects Ralstonia solanacearum.

18. The bacteriophage of claim 17, wherein the Ralstonia solanacearum is strain MAFF107624, strain MAFF211266, strain MAFF211270, strain MAFF211543, strain MAFF301859, strain MAFF311644, strain MAFF730103, strain MAFF730131, strain MAFF302745, strain MAFF311632, strain MAFF211536, strain MAFF331041, strain MAFF730139, strain MAFF211280, strain MAFF211533, strain MAFF211468, strain MAFF211516, strain MAFF311101, strain MAFF311102, strain MAFF211479, strain MAFF211471, strain MAFF211483, strain MAFF211484, strain MAFF211486, strain MAFF211272, strain MAFF211276, strain MAFF211278, strain MAFF211490, strain MAFF211492, strain MAFF211497, strain MAFF211476, strain MAFF211414, strain MAFF211429, or strain MAFF301558.

19. A composition comprising the bacteriophage of claim 9 and a pharmacologically and botanically acceptable protein stabilizer.

20. The composition of claim 19 which prevents wilt by Ralstonia solanacearum when applied to a plant.

21. The composition of claim 19, wherein the bacteriophage is present at a concentration ranging from 103 to 1012 pfu/mL.

22. A method for treating or preventing bacterial wilt disease comprising contacting or injecting into a plant or plant growth medium with the bacteriophage of claim 9.

23. The method of claim 22, wherein the plant is infected by Ralstonia solanacearum.

24. The method of claim 22, wherein the plant is infected by Ralstonia solanacearum strain MAFF107624, strain MAFF211266, strain MAFF211270, strain MAFF211543, strain MAFF301859, strain MAFF311644, strain MAFF730103, strain MAFF730131, strain MAFF302745, strain MAFF311632, strain MAFF211536, strain MAFF331041, strain MAFF730139, strain MAFF211280, strain MAFF211533, strain MAFF211468, strain MAFF211516, strain MAFF311101, strain MAFF311102, strain MAFF211479, strain MAFF211471, strain MAFF211483, strain MAFF211484, strain MAFF211486, strain MAFF211272, strain MAFF211276, strain MAFF211278, strain MAFF211490, strain MAFF211492, strain MAFF211497, strain MAFF211476, strain MAFF211414, strain MAFF211429, or strain MAFF301558.

25. The method of claim 22, wherein the plant is a member of Solanaceae, Lamiaceae or Zingiberaceae.

26. The method of claim 22, wherein the plant selected from the group consisting of tomatoes, eggplants, bell peppers, or tobaccos of the family Solanaceae; Perilla frutescens var. crispa or Perilla frutescens of the family Lamiaceae; and ginger, turmeric and curcuma of the family Zingiberaceae.

27. A bacteriophage tail protein comprising a host recognition site for Ralstonia solanacearum shown in SEQ ID NO: 1.

28. A base sequence encoding the bacteriophage tail protein of claim 27.


SEQUENCE LISTING (3 sequences)

SEQ ID NO: 1 - protein, 551 aa, Unknown
  MISC_FEATURE: Bacteriophage RKP181 host recognition domain containing C-terminus of the tail fiber protein
KTWRDETLTARDATFTARDTTLGYRDTTLTYKNAAEAARDAGFAARDVALAARDTTLGYRDTANQHRIDAQAARDLALQYRNEAEAFKNTASDKADIVLTIEATANSAKAEAQAATAATTGLMDNDLQLFVNGTQRRDIKVKAGKHDAQGVQRAGVRFTDVDTIAVERYNQTTGVIIDTPVKIDQATGDVVLASKKIKGVTTPTANDEAANKSYADTKLPFVGGTLTGSLFVSGATDNAGAFRVTADLGGAWVDWNAARKYPLQIDCPASSAAYGGIRWTRWGARHIAAIDAFDNTGGAPVIVMHLGDQNNAWSFYKDNIYRGYNGGYVWGSWNFNPASKANADWITDVGIEGNDIRRPYVRRASDGYIVRMPRNFSGNALEVGWDGGLRWYVDGVHQGAMVSDTNWRGAFHNDAGRIGRGGDWSIGNGYFDVVIDGVSYGVPFNPSDARRKENIVPSTEDAIEKVKQIKFYSFNFKADGLMDSKKVHQTGFIAQQLQGVDPALVIGDDNPDMTLSPDVNSLLSLALKSIQQLDAKIAVLEETILALKGNP

SEQ ID NO: 2 - protein, 711 aa, Unknown
  MISC_FEATURE: Bacteriophage RKP181 host recognition domain containing C-terminus of the tail fiber protein
  MISC_FEATURE (1)..(148): a domain corresponding to the T7 tail-attachment domain
  MISC_FEATURE (161)..(711): the host recognition domain containing C-terminus
MAAPTTVKVYPLNNVVKDFPIDFDYLAREFVVVTLLGNGRRELVQNSEYTFLSATQIRTTATWGPAQGYDNIEVRRVTSAENRLVTFNDASILRAADLNLAELQTVHIAQEARDLVSDALGTNDDGNLDARNRRIVNLGNPVDPQDAMTKDYYDSRLGEAKTWRDETLTARDATFTARDTTLGYRDTTLTYKNAAEAARDAGFAARDVALAARDTTLGYRDTANQHRIDAQAARDLALQYRNEAEAFKNTASDKADIVLTIEATANSAKAEAQAATAATTGLMDNDLQLFVNGTQRRDIKVKAGKHDAQGVQRAGVRFTDVDTIAVERYNQTTGVIIDTPVKIDQATGDVVLASKKIKGVTTPTANDEAANKSYADTKLPFVGGTLTGSLFVSGATDNAGAFRVTADLGGAWVDWNAARKYPLQIDCPASSAAYGGIRWTRWGARHIAAIDAFDNTGGAPVIVMHLGDQNNAWSFYKDNIYRGYNGGYVWGSWNFNPASKANADWITDVGIEGNDIRRPYVRRASDGYIVRMPRNFSGNALEVGWDGGLRWYVDGVHQGAMVSDTNWRGAFHNDAGRIGRGGDWSIGNGYFDVVIDGVSYGVPFNPSDARRKENIVPSTEDAIEKVKQIKFYSFNFKADGLMDSKKVHQTGFIAQQLQGVDPALVIGDDNPDMTLSPDVNSLLSLALKSIQQLDAKIAVLEETILALKGNP

SEQ ID NO: 3 - DNA, 39455 bp, Unknown
  MISC_FEATURE: Bacteriophage RKP181 host recognition domain containing C-terminus of the tail fiber protein
  DTR (1)..(239)
  ORF1 (404)..(634): encodes hypothetical protein
  ORF2 (1304)..(1504): encodes hypothetical protein
  ORF3 (1769)..(2011): encodes hypothetical protein
  ORF4 (2022)..(2441): encodes hypothetical protein
  ORF5 (2519)..(2764): encodes hypothetical protein
  ORF6 (2820)..(3002): encodes hypothetical protein
  ORF7 (2999)..(3178): encodes hypothetical protein
  ORF8 (3175)..(3495): encodes hypothetical protein
  ORF9 (3492)..(3857): encodes hypothetical protein
  ORF10 (3870)..(4049): encodes hypothetical protein
  ORF11 encodes hypothetical protein
  ORF12 (4320)..(4586): encodes hypothetical protein
  ORF13 (4651)..(5007): encodes hypothetical protein
  ORF14 (5058)..(5465): encodes hypothetical protein
  ORF15 (5469)..(5570): encodes hypothetical protein
  ORF16 (5567)..(6499): encodes ATP-dependent DNA ligase
  ORF17 (6496)..(6615): encodes M56 family metallopeptidase
  ORF18 (6768)..(9365): encodes DNA-directed RNA polymerase
  ORF19 (9385)..(9831): encodes hypothetical protein
  ORF20 (10012)..(10257): encodes hypothetical protein
  ORF21 (10254)..(10553): encodes hypothetical protein
  ORF22 (10616)..(10921): encodes hypothetical protein
  ORF23 (10914)..(11609): encodes hypothetical protein
  ORF24 (11590)..(11763): encodes hypothetical protein
  ORF25 (11831)..(12496): encodes Putative ssDNA binding protein
  ORF26 (12499)..(12966): encodes T7-like phage endonuclease
  ORF27 (12968)..(13438): encodes lysozyme [Peptidoglycan recognition proteins]
  ORF28 (13545)..(15230): encodes DNA primase/helicase-like protein
  ORF29 (15240)..(17348): encodes DNA polymerase
  ORF30 (17398)..(17775): encodes hypothetical protein
  ORF31 (17798)..(17998): encodes HNS binding protein
  ORF32 (17995)..(18879): encodes phage exonuclease
  ORF33 (18901)..(19212): encodes hypothetical protein
  ORF34 (19226)..(19591): encodes hypothetical protein
  ORF35 (19594)..(19896): encodeshypothetical protein
  ORF36 (19901)..(21496): encodes Putative head to tail joining protein (collar)
  ORF37 (21557)..(22381): encodes Putative capsid assembly protein
  ORF38 encodes Putative major capsid protein
  ORF39 (23560)..(24138): encodes Putative tail tubular protein A
  ORF40 (24141)..(26507): encodes Putative tail tubular protein B
  ORF41 (26615)..(27079): encodes Putative internal virion protein A
  ORF42 encodesPutative internal virion protein B
  ORF43 (27663)..(29846): encodes Putative internal virion protein C
  ORF44 (29865)..(33749): encodes Putative internal virion protein D
  ORF45 (33833)..(35968): encodes putative phage tail fiber protein
  ORF46 (35968)..(36294): encodes Putative uncharacterized protein
  ORF47 (36294)..(36506): encodes Putative lysis protein
  ORF48 (36503)..(36775): encodes Putative packaging maturation protein A
  ORF49 (36798)..(38579): encodes Putative packaging maturation protein B
  ORF50 (38795)..(39007): encodes hypothetical protein
  DTR (39217)..(39455)
tctcatagaatgagccgctgggattacccccatagggggtccacttgggccactggggcaggcaggaggcccccccgggtgcccctctcggcactccctgaggggaaccggcagggtcaacccactggatttaacataatggaccttatgcgcaatccctataggtcacattgagggtactgataggggctctctatgggctcctctggggtgataggagggctctatgggtttccctatctgtaggaccctgaggggcaccctgctatgccctaccctgaggggcgcccctttgggacacctgctagggaccgtgctgtatcccttgccttacctgctatgggctctatactacagggtgaccctgagggtgtcaatccctgagggtcacattccgggagacctacattccaaccagtgcggccctaatgcttgtcttcgtgtagaccgcaccgtctggcgtcttggtcatcccgtatgcttgcagcatctcaaacagtcgggcggcggcttggtccttggtccggtaagccttcaggcattgctgcgcctgtctatatagtccttcgtcgttattgatccagagggagacattccactgttgccagttcttatgcccattgtatccagccatgatgcactccttgagggttacacatgagggggactacaggacccatgataggggctatctgtcattgcgtcaatagggtaaacccttgggcgagtgtatttcttgtctcagtgggcgacaatttcccgtgcctctatatagggcgagagtcagcccattaacacacactaaagaaagtgcatgttgcctattgacggcctaccgattctcaggcaagattcgtctcacggtgttgcaacgcagcgccggaccgggagaccgggacgatctttaagaatctgaccaaatgactgttgacagacacggttacatgctgatacagtgtgtaccagtcgcaacgcagtagcccctgagagtcaaagccgacgttaaatgctagggttgaacagtatgtgtccctcctttccttgattgggcgagtgatacgcgacaacgtgttagatgcagcctaggacagattcctgagaatgcaagtagagattgacaaagcgcagcgaatactgcacaatgcggtctcagtagtaacggaacagcagcaacgttccggcagtaacagaaagtgattgacaaggtgaagcgaagtatgcgacactgtagtccaaggtagcacaagacgcaacactccgcaagggtgttgtgatgatcgaacgaagacaacggaacgcacacatgaacccgatcaagcgcgcactgcaccagtaccgcgaagcggccaagatggtcctcgccaagcacttcggttacattgtgtggtcggctgagtaccgcgcaatacacgtctccttcaccctcaagggtgccatcgagtggcaagcctgctacgacgatgcggtgatcgtggcggcccagcgagtggtccgtttcgcctgataccggcaagggtatcggttgacagtgtagggcgccaccaatcgcagcacggcgcatgggcaacggcccagtaggaaacccgcatagcgacgggtaggacccctgcaaggggaaccacggggcgaccggctggggcgtccgatggaataccagcacacaattcgggttgcgtagggggctcagagtgtcccaatgcgtagtggatacggtgacgattcatcctctgtcctagtgaaacgagctaacgtgtaagacgcgaaccgaatggctaaatccgaagggctgacccgcaagggtcggtccttccagtgtaggcattcccgcccaaaccgcaaggactttttcatgtactacctcatcgagtatgacgatctgtgcaaggtggatcacgcaagctgggtcgtcaagcgatacgaggagtacgcggacataccctccgcgctcgaccgctgtggcgaactgatcatgcaaggccatgccgtacacatccaacccctcacccgctaaggactcaaccatgagcaacccctttgtcatcatgtccgcattccgtcaagggctctcccaagaggagaacctcaagcgtcaccactcgctggacgccactgtgggcaccatcgtggggcaacaccctaagctgaccctcggctcctatgtggaggaggaaacgcaacgcccgggcatcgagttgggcgtgcgcatcgagccaacagcacaaaccagctttgcaaaggtagtgcaagtcgctctccggcttgccgagcacttcgagcaagattcgattctcgttgtcgatcacggcgggcaagcaacgcttgtgtaccgtgacggacgcgccgatgaaggagtcggaaattggacgaaggtgtctcaagctgaggcattgcggtctccgtactggaccaacatctcgggatcgtactacaaggccgtctaagttgcaacgcccaagggcatccgatcagggtgtccttcaacgtttcaaccgcaaactttccgcaaggataccacgccatgagcgaacaaacaatacgcatcaacgttgcgaagaagcaaccacagcccgggttcggcggcaagcctgtctaccgccaccactgcgccgtccacctcgactggctctccgatctggacaaggtacatgaggtagtgcagcaactccgcgtggccttcccggtgggcgagtaccacatcacggtcacccgctgggtcctccgtggggaacacatcaaggtcgaaggctgtgacgacgaccgctgaggtcctgcaagggtggctagtgtggcgcgatcagggatcgtccggtgggtggaccgtgggggtctacctgtgggaacccgatgcgctgcgctacatccgggagaaccggggcgaacgcctcaagggctggtggcccacgacctactcccgcacctaccgccaccgcgctgtgcagtgtgtccgcatcgacgggaccctgcatgtcctccaaccctgtcacgagaacccgccgatatgagcgcaagtcaaggccaactggtcgaggtggttgtcaccctgaagggcaaccgccaagagacctacttcatcaccggattgccgaacgcaaggggcgtatcccgcctgttcatgggcaacccgcaagtcctgaaagttgaactgctgtacaccggagaacgaggagcaacccgatgatcgtcaacgctgcaaccttccgcgccatcctgcgcaacatccctgccaacgcgctgcgccccatcatcgagggcgatctgtcgcgtgtcgaggtcaaggacaccacatggctccatgcgcccatcttggggaccttctcgggcaccaaggacggcaacgtggtggtcaagttctggggcgacgattacccgctcctgtactcgctggcaaccgagctttacaacaaggccggggacatcaccatcgtgcaagccctcgcgtggatcgcgttccggtcctacgaggaagcgttcgcaaccagcaaggaggcagcatgagcgaagccgtccagtacctcggccacggcccggagggtggcattcaggaacactccaagggcaagctgcatcccttggtctgcgtggtcaaggaaccgcccgccgatctgatcgaccagcgcaagctgacctactggatcgacccgcgcaagggcggcaaggacgttaccggcgaatggcgcgatccgttcttcgccctgaagtacgcaatcctgtattcgtggggatacaagttcgacaccgagcgcccgctgggcgtgggtctggaactgttcgccgctgccgagggccgcaccgtggattccctgtaccgcgaggcccagaacatctccgccgccgagcatggccgtggattcgtctaaggaggaggacgcatgaaccgcatggaccgcacaagcctgtcccgcttatgtctcgtgctgttgctgtgcgtgctgatcctggccctgctcgggctgggcggctgcgcagtcaacaccgtgaccatcaacaaccacatcgaggtgcatgacagcgccatcgacgtgcaagccaaccgcctcacccgcatctaacccgcaacctcaaggagataccaacatgctgcgttccttcaatgccttcatcgcccgccgctggaacaacttcaccacggctgtggcgttcgtgatcaacctgccgatcatcttccacatcctcgtggtgctggtcgcgctgctgatcggaatcggggccgtggtctggtccgtggtcgagtcgggctggctggtgatcaaggccctcgccaccctcctgtgggcgcccttcaaggccatcttcgtggtcggtgcggtggtggccgatgggtggaccagcgcggggttctgagcgtggccctgtcaaccgtccgcgctgtcaagttctggcggcgcaaccacgaggggaccctcaaggcgtcccactggcagtacggcttgggtccctgcggcgagcatgtggggaccctgcgtgctggcttcgacggtgactggctggtgatccaccagtacaccgacaagaccgagaagctgttccatttccatgtccgtgacatcgcgggcaaggtcgagttcctgctgttcgaggagccgtgaaatcagggggcatcctcaagggtgcctccgagtttcatcccaaccactcaaaggagagtgaaccatgcgtcaatccgccatcgacaccagcctcgcccccgaactgaacgcccgcaacgaggcgagcagcacgtacccgttcgaggtcgcctatgtgggcgagtactggcgagtgcaggacaaccgcaccgggggcctgtcttcgacccgccaccagctttacaagagcgccgaggacgaagcccgcgaactgaaggccaagcagcaggccgacaacgcggccaaggttctggcccaagccgaggcaagccccgctccggtcgctgagcccgaggcaacgcaacctgccgctgaaccggccaagccgacccgccgccgcaaggcgaaggccgaggcgattgtcgccgccgaactgaagtaagccacccgctcaatcctggccctacgggggccaccctttaaggaaccctgatgattctccgcaacgctcccgccctcggcttcacctttgctgaggcccgcgccgctcgcctgttgtctgcctcctcgcctgaagcgtgggccgcaacccagcagcgcaacgccatgatccacacggccaccgatccggtcctccgcgcacaagccgacgagaaggcgcgcaaggccctccgggactccctcaagaaagcgatggggatggccgcaacggggcatggcccggtgggcgtgttcaagcgggggaccatccgggtgtcccgcgagggtgtctccgtgggcatccctgcgcatctggtcgaggaggtctcgcccgagttccgcgaggaggtggccgatgctgtgcccctcaacgccaccgcgagcaacacccgccaccacaacccgaagcgcaaggactgagccatgagccacgacaaccaaggattcgagaagtcccgcaacaccaagccccgccgcgactgggcggacctgcaagctgcccgcaagcgcaaggagaagcgatgagccaagcccactacctgaccaacggttaccacgagttcaagccacggggcgctgtcccgttcgaccgcaagggcatcgccgctgccatcgagcgcactggctatgtccacatggcctacaaggtggacggcgtgcgggtcatcctgcaagtccacccggatggccgcgtgacctgcaccagccgcacgagcaaggtcctgcccgcgctgcgcctgctgggcgccgaccctgacctccgggagaggctgcgcgagaagtacccgcgaggcgtggtgctggactgcgaaatctcagtcgtgggccggtcctttcaagagggctgtggcgacctgcgccgcaaggtgccgattgacccgacctcgctggtggtctggccgatcctccagtacgcctacgacgaggtggtcacgggtgtctacgccccggtgacgctgggtgtgcgcatggcctacgccctgacgctggccgaggaactgtcgccgctgctgggcttcttggtccatgacctgcgcatcgggaacatcaccagcgtggacgacatcgagccccagttcgaggtggcccgcgccctgagttacgaggggctgatcgcctacgacccggccaacgatgtcttcgacgggggcaagcgcgtgggctggtggaaggtcaagcccgagtcggacatcgacggcaaggtggtgaactggatcgagggcaccggcaagtacgtgggcatgatgggcgccctcacggtcgagtgcgaggacgggaccatcacggacgtggggaccgggttcaccgacgatcaacgcaagctgggccgcgagtactggcttggccgtctggtgcaactgacgttcatggaacgcaccgacgatggcaacctgcgccaccccgccttcgaccggatgcgcgacctcgactccgcaccggggctgaagtcatgatcgactggcccctcgtggggctcctcgtgctggccgcgctggtcctcgtgggcgtctgcctgacctacctcgggcggctcattgaggaggaggtctcggaccccttcaacgattaacccacactagagcacagtgtcgaatcccttaggtgcgattaacccgcactatagggggttcctctctgttctcctgaaagataaaccttaaaggtccctgagaggggttcctgacaaggtagacataaacaagattaccctataggaaccctatgagcgatgctttgcaaaccctgaccagcctgtacggtgctgatctggctgaggcgcaacgggccatcgaggcggaagcctacgagattggtgcccaacgtttcctcgctgcgatgcaggccaaggccgagcggggcgagggggcggataccaaggtggcccgcccgctgattgccgacttgatccccaaggtggaggcagcaatcaacgccttcatcgaggtcgcattctccggcaaggctggcaagaagcataccgccgccaagctgatggcgaaggtcaactccaaccgcgtggcgttcatcgccctgcgtgtcgtgctgaactccctgtcgcaagtgagcggcgggtcgggtggcaagaagcccgcaaccctcgttgctgtgtcgatgcagatcggccatgacattgaggacgaggcccgcttcggacgcatccgcgacgacaacgagaagcggtacaaggacaatattgcggtgaacatcgccaagcgttccggcgaccacttcaagcgagcctatgctcgcgctgtcgaggttgcgatggaggacgcgggcgagttgcaggagtgggatggctggtcgaaccaagaccgcgccgctgtggggctgaagctggtcgagttgatcattgaatcgactgggctggtagaactgcgccacgagcacaagggcaatccaaagttgcaccgagtagtgctggaaatcacggacgcatacgcggagtggctgtccaagcggacgcactccctcgcggacatgaccccggcattcgccccgtgcgtggtccctcccaagccttgggtcgggaaccgtggcggtggctactggttcaacgaggacaagtctccactcatgctggtgcgcgggtctgtccgccgcaaccgccgctacaaggacgtggacctctcaaacgtgctggccgcgctgaacgcgatccagaataccccgtggacgatcaacgccaaggtcctggccgtggccgaggaggtggcgaagtggcccaaccctccggtcaagaagatgccctcccacgtcacgctgcaaaagcctgaacggctggaaggcatggacgaggacgaggaggtcctgcgcaagtggaagaaggacgccgcgctggcctaccgcaaggagaaggcccgccgctcgcgccgctaccaactggagacctcactggtgcaagcccgcaagtacgccgggttcgagcgtatctggttcccctactctctggacttccggggccgaatctacgccgcgactaagttctccccgcaagggcaggacttggacaaggcgctgctgctgttcgcggaccctcccgagattggcgaggacggggcgttctggctgcgtatgcacctcgccaacaccgcaggcttcgacaaggagaccttggacgaccgaatcaagtggacgcacgacaacgagcaactgattctggagaccgccgccaacccactcgacaatctctggtgggccacggatgcggactcgcccttctgcttcttggccgcgtgcttcgagtacgcagcgtggaaggagcaaggccccagctatcgttgcggtctggcgatagcgttcgacgggtcctgctcgggcatccagcacttcagcgcgatgctcaaggacgaggtgggcggcaaggccgtgaacctgatcccctcggagaaaccctccgacatctaccgcatcgtgtccgacaaggtgaacatcgtgttgaaacgcgatgctgccgatgggtctgacgactacacggacaccaaggtggatgacgacaccggggaaatcaccgagaggccccgaagggggacccgctcgctggcccgccactggctgaactacggggtgaaccgcaaggtcaccaagcggtcggtcatgacattgccctacggctccaagaagtacggattcacggaccaactgttcgaggacatcgtgcaacccgcaatcgaggagatgggcgaggacgtgttcccggccccgggcgaagcctgctcctacatggcgggcctgatctgggacgcgctgggcaccacggtggtggccgctgtcgaggctatggcatggctccagaaggtggccgctgtgctggttgccgagcagatgccgtgccactgggtcacgcccgctggcttcccggtctggcaggagtaccgcaagtcggacacgcatcggatcgacaccatgatctgtgggaacatccgcgtgaccatgaccgtcaacaagtccgaggcgcagaccggcccgaagccgctggaccgctacaagcagcagaacggcatctcgcccaacttcgtgcatagcatggacgcaagccacatgatgctgacctgcctgtcggccagtcgccaaggcatcgagcacttcgccacgatccacgactcgttcggcaccgcacccggccacgctggcgtgatgttccggacggtgcgtgaggtcatggtcgagacctacacctcgcaggacgtgatccagaacttctacacgaccttcgactcgctgctgtccccggacgcacgagacaagattccggctttcccggagaaggggtccctcaacctgatggacatcctcaactcgcagtactgcttcgcttaactctcaaggagaacaccacatgcaatcgaccatcgcccaagcccacgccctgaacaccccggaagccaacctgatgcaccgcatggcccacattcagcgcatggaggaactggtccatagcatcaccgaggagggcacccaagcccgcgtcgatctggaagcccacatcgacaacctccgcacggaaatccacgcagtctaccgcgccgagaagcacgaccagattcgtgcccgccatgcgtatgcccgcaaggccacggtcgaggcgctgcccgccgaaggcccggtctaccaagcgggtcccctgtcggacaccttcgtggtctcctccggcgaacgcaccgaggtcggcgtgctggtcccggtcctgcactcgcccatcgacggcgaaccgctggtcccggctggcctgctggcccgcgcatgggccgctgtgaagggcgtgttccaccgtgccgcgcactaacccttcgaggatcacccacgcgggcaccacgctggtccttgtctataccgacgacgaggatcaagcgtgcgcccagtgcgcgttgaccaagctgaagaactgctccgcgatcccgggcgccgcaaggtgtgcccacaaggatcacggcttctggattcccctcaaggagataccccgactatgaacaagaagcaagcccctgaacacaaggacgtgaccggcaaggtcatcaagctgggcgataccgtggcgttcgccaaggctgggcactgcgcgctgtacgtgggccagatcacgaagattcacccgaagctggtgcgcgtggcctgcacgacccggcacaagcactacatgagcggcgcggttgtcgagcagacgaacgagtacgaccgccacccgctggacaccgtagtggtggcagcatgaccgtgggccgacactgcaccaactgcaagcatcacatcgacggctactggaatcaggagtgccgcaagttctccaaggaggagacccgctgggaacccgtgcagggtccctacaagtaccgcaacgctgtgacctgcgacgacgcccgctcgctgaacggcctgtgtggtgtgcaggggaaatccttcgaggcatcccgctgggcgaccctcaaggataagttccgctgggtctacctgctggcgcccgtggcgggtctcatcgtggggcacatcctggcccgagtgttcgcctgattaacccacactagaggacaccccacggcaccccgctgtgggacctcaaaggagatacgactatgacgcttgagcttcgccgccgcaacaacggccaaagcgtggtgctggttcccgactcgcgcagcatcgactgcgaacggaaccgcaacggcaaccgcaaggccatcgcaacgttcaccccgccgtacacccgctcccgtgtcgagaagtacatgggcaaggtggtgctggtctccccggcactgctgcaacaggccctcgctgaaaagcgtgtggcactggaccacgatccgcgccgcttcctgaaggcccgcgctgccctgtcgaacgcatgggctcgcgtcaaggaggtgttccgtggatgacgcaacacgccaacgcgctctggaatccgtgaagggtctcggcctcgccgctgaccatccgggcgcgggcaaggacaccgccttcgcatcgctgcaagccgccaacccggggaccgatttcgtgaacgtcaagttcgcggatgccctgaccgccgaggtgcgcgcactgttcccgaaggttacggacgacgagttcaccgagattcgcaacgacccgaagttcaaggacctgccgctgtggatgtttgcgatccacaacatcgcccgcaccgagcaggggctggactacgtggccttcctgaagcacgagcaccccgaactgatccgccagcgcatgtcgatccgtcagcatctcctgatctacggcaccgagtacgtgcgtgagttcaagggcgacgaggaccgctggctgaacctaggcgtggcgaaggccaacgaggtcgccgcatcgggcaaggtcccggtgatcacggacgtgcgcttcgtgaacgaggccatcaagctgaagcaggccggttacgacatcgcgcacatcgctgccgatggtctggcgaacgccgccctgtccgccctgaccggcattgccgaggggcacctgaaagactgggactttgacctccgcgtgaagaacgtgtggggcaagcccgagaacatgggagtgcaattcaatgccaagtaccgctggtaaggcactgaaggttttccgtgtgcggtacgccctcgaagggaccccttacgccaccgctgtggaagtcaaggcggtctccatcgacgctgcctatgaggccgccgaggagatgtaccccaactgggaaatcgaagcagtgatccaagtgggataacccacactaaaggacaacatcaagggaccctccggggtcctttctcatttcagaaggagaatcacacatgagcaacgcacccgcacaagccgacaagccgaccacgctggtgaccccgctggcgaccgtcttcggcttcgtcaacgtcaccaagccggacaccaagtacaagcccgagggcgagtacaagattcgcgtgaaggtccccaaggaggccgcgcaggacctctacgagaagctggccgctcaagccgagcgcaagctggaggagaccatcgcccgcgccaagaaggacgccaagttcaaggccagcctgaagggcaaggcaccgaaggccgctgacctcccgttctacgaggacgaggaagacggcacctacgtgttcaccttcaagagcaaggccagcttcgtcagcaagaagccgggttcggaaggcgagaccatcaaccgcacggtcccgatctggcaaggcaacaagcgcctgaagccggaggaggtgcccaagtttggcgagggctcgcaagtccgcgtgtcgttcgtggccgctgacttcttcaccgctgccgtgggcgctggcatcacgctgcgtctggaggccgtgaagctgatcaaggcagtcgagtacaccggcggtggcagcaacccgttcggtgacgaagatgaaggcgactaccaagagtccaccggcaacgagttcggtgatgacgaaggtggcgtggacaccgagttctgatcatggcaggccgtccgttcaaacgagcgggcggctggggttcccataacccatcgacctatcgcagcggtctggaggacaagatcgccacgcaactggaggggaagtcccttccggttgtgttcgagcagttcgaggtgaagtatgtaatcccagcctccgatcataaatacacgcccgacttcgtgctccccaacgggatcatcatcgagggcaagggaatcttcgacgcagacgaccgcaagaagcatctcctcatccgtgagcagcatccggacttggacattcgattcgtgttttcgagcagcaacgcgaagctatacaaaggctcgcctacttcctacgcgaagtggtgcgagaagtacggcttcaagtttgcggacaaactcattcccgattcatggctccgagagcccgggaccggacgttcgctcgtttcactgacccgaaagaaaaagaaggaggcatgacatgctccctaccccgtaccccggtctcccgccgatgaagaagcgggccacgaccgacctgatcgtcattcactgcgcagacaccaagcccacgatggattggggcgcacgcgacattcaccgagtccatgtggtcgagaaccactgggccgcaatcggctaccacatcgtcatccgccgagatggcaccgtggaaggtggccgaccactcgacgctgtgggcgcgcacgctgcacaggtcaactcccactctgtcggtgtctgcctcattggcggctacggcggtaaagccacggacccgttcgagaagaacttcacgaaagaacaggccctgtccctccttgtcatcctcaaggaactacgcgagaagtacccgagcgcggccatcatcggccaccaagacgttcagggttccggcaagacctgcccgaacttccccgcgaaggcatgggccgctgaacacatccagtaggccagcatcaagagaaaccccccgagggtccgtaatgggctcccgggggttttttcgttcagggccattaacccacactagtggaccaactctgacggagatgatgatggcacgacaagacaatgaccgcgacgaatcatcggacttcgttcgccacgtaccgtgcgacgaatgcggcagcagcgatgcgaactcgctttacaccgatgggcaccagttctgcttcgcctgcgagacctacgtaccgggtgatgggcaggaactcccagccgggaagaagggccggagccagcgcgccgagggcgtcctgagcatgggcgaggccaacggccagtacaaggcactgcaagcgcggggcatcaccgaggagacctgccgcaagttcggctactgggtcggcaaggtgaagggcaccccggtgcaggtcgctgactaccgggacgtgaccggcgctgtcgtcggccagaaactccgagacccggagaagaacttcaactgcacgggcaaggtcgcacgcggccacctgtggggctcccacttgtggagcggcaagggcaagatgatcgtgatcactgagggcgagattgactgcctctcggtgtcgcaactgcaaggcaacaagtggccggtggtctcgctccccacgggggccaaggcagcggccaagtcgtgcgccgagaactacgagttcttggatggctacgaccagatcatcctcatgttcgacaacgacgagccggggcaggccgcgatggcggaagctgctgaagtcctcccggctggcaaggtgtttatcgccaagctgcccctcaaggacgccaacgagtgcctactcgcgggcaagggccaggatgtcatcaacgcgatctggaacgcttccccgtaccgtcccgatggggtggtcgctgcgcgtgacctgatcgaccgcatcaagacccgcgccgtggtggagggcatcaagttccccctcgggcagaccctcaacgagatgacgctgggcatccgcgagggcgaagtggtgatgctcacctcgggctccgggatgggcaagtcgtcgttcgcccgtgagtgcgcctatgggtggggccgcatgagtggcctcaaggtggggatggcgttcatcgaggagtcggtcgaggagacctgccttgacatcgctggcttgcacctcgggaagcgcatccgccagttccccgatgccgtgaccgtggaggtccgggactcggcgctgaacgaactgttcaacaacgacacctatcacctgtacgaccacttcggctcggctggcgaggactcgctgctgaacaagctgcgcttcatgatcaccgtgctgggttgccagttcatcatcctcgatcacctctccatcgtcgtgtctggcatggacgagtccgaggacgaacgcaagaccatcgaccgcctgatgaccaagctgaagactctcgccaagacaacgggcgggcgcttcgtggtgatcagccacctccggcgcaaggactccaagagcacgagtcacgaggagggcgggcagatcagcctgtccgaactgcgcgggtccggggccatcgcccaactgtcggacaccgtgatcggcttcgagcgcgaccagcaaggcgaggacccgaacctcgtcaccatccgcatcctgaagtgccggttcactggcgacaccggggtggcgggcttcctccgcttcaacaaggacaccgggcgactggtcgatgcgtggctcgaacagggcaacgacttcgggccggatgacggcggctccaatcctgatgaccccttttaaggagaaccaatgattcaagagaaattcccaaaggtccgcctgttcgacatcgaaacggacgggctgatggagtactcggtgatcccggggctgacggaatcgtcggtggtgtccaaggtgcattgcatcgtgatccacgactatcacgccaactggtatcaccgctaccggtccgatgtgccgggcgacatcgagcgggcgctggacgtgctcatggagtcggaccttctggtggcccataacgggatcaagtacgacatccccgtgttggaactcctgtacccgcacttcaagatcgaccgccgcaaggtgctcgatacgctggtcctgtcgaggctcctgcacgccaacatcaaggacacagacaacgcgctgctgcgccgtggggaactcccggggaaactctacgggtcccactcgctgaaggcgtggggctaccgcgtgggcgaggccaagggttcctacggggaaggcgagggcgatgtctgggccgagttcaacgaggagatgctggagtactgcgagcaggacgtggtggtgacccgaaaggtgttcgaccgcctcctggcaaactcctactacttcagcgaggacctgtcctacacccagtacgcagtccgccttgagcatgacgccgcgtgggtgctggcccagcaggaacggaacggcttccccttccacgagaagggcgccgctgcgctctacgcggaactgtcaggcaagcggaacgacatcctgcaacgggtgatggagaccttcgggtcatggtatgtggcgaagggcggcaaggaagccttccggcacccgcgcaccggggccaagctgctgaagtacccgaacgtgacctacccgaagaccggggacatctacacgaagtccggcaagctggcgaagatcgactactgcaaggaccggccctacacgcctgtcgagcatgtggtcttcaatccgggctcgcgccagcacatcgccaaggtgttgcaggagcgcggctgggtgcccgaggagttcaccgagaccgggcaaccgaaggtggacgaagaaacgctggccgaagctgccgagcgcctgcccgagcagtaccgggaggacgtgaagctgatcgcggaatacctgctgctgatcaagcggctgggccagatcgcggaaggcgacaacgcatggctgaagctgtgcaatcgcgggtacatccacggctcggtcaacccgaacggggcagtgaccgggcgggccacccatgccttcccgaacatcgcgcaggtcccctcggggactgccctctatggccccgagtgccgcgccctctttggggtcgcctacgtgcgatcccgtccgggctgggagaaggcggtccaagtgggcaccgatgcgtccggccttgagcttcgctgtctggggcacttcatggcgaagttcgacgggggccagtacatcaaggacctcctcgaaggcgatattcactgggtcaatgtggagtccttggggttcgtcccgaggggcaccaagcggatcaaggaaggccccggccacgaggagcacgacaagttccggggctacgcgaagaccttcatctatgcgttcctgtatggcgctggtgacgagaagatcggctccatcatcccgggcggggacaaggcagtcggcaaggacctcaagaagaaattcatggagggcaccccggccattgccgaactccgcgcactgctggaggagatgctggtcgagcaacagaagtgggtggacggcgtggcgcaggtcaagtggaagcgccgctggatcaagggcctcgacgggcgcaaggttcacgtccgctccccgcacgctgcgctcaacaccctgctgcaatccgctggcgccctgatctgcaagtactggatcgtggagaccgagcggattctggtcgaggagatgggcctgaagcacggctgggatggtggcttcgcctacctcgcatgggtccacgacgagatgcagatcgcggcccgcaccccggagattgccgagaagatcgcggaagcctcgcagaaggccatgcgcaccgctggtgacttcttcaacttccgctgcctgctggacacggaatcgaaggtgggcgaccactggggcgattgccactgaccgattaacccacactagtggacaacacattctgaaaggaggccaccacatggcacgttcgtttcaactcaacgtttccacccgcgtgaaagtggtggtccccgattcggagatgccggaagtcgtgaaggcattcgaggaactgaaggtcatcgctgcgcaactgcgcgtgctggtcgagcgtgaggacatgccgaagctgcgctccatgttcgcccgcaacgaggacatcccggatcgtgcggtcctgaagatcgcccggggccgactgcgcgccttcgccaacatcggggaggacctcgactcgttcatcctgttcagcgtcaagctgaatctgaaggaccagttcaaggacaacctcgacggtgatgccacgctgtcgttcagcccgcccaagttcgagaccgtcaaggtctacggccagtaacccatcaacacaggagaattacatgaacgatctgatcaaagtctgcaccgccatccaccaagacccgaaggcgttccagtccgactacgcccgcaagcacgcctaccacatcgcggaagctgcgagccgtggcctgatcacctgcatcacgatgggcctgaaccgtggcaagtggttcgtgacgcaagagggcctgtccctcgtcaagacgcagcagcaatgagcgaacagcgcctcggtctcctgatcgacgctgacttcctcgctttccaagcagcggccaacgccacccgcgtggtcgagtgggaagatggcgtcctgacaacgtgggcgaacatggaggattgcatcaacgccttcatgacctcgctggaagcactgacctcgcgcaaccgccgctggtccaccgcgaaggtgatcatgtgcttcaccgacgatcacaactggcgcaaggacatcctgcccagctacaaggccaaccgctccggcgtgggcaaggggaaacccatcgcctactggaagctggtcgagtgggtccaccagaacttcgagtgcttcgtgcgcccggggctggagggcgacgactgcatgggcatcctgtccaccaagccgtccctcgtgggatgcacccacacggtcatcgtttcccccgacaaggacttcaagacggtccccggggagttcttctggatgaccacgggggaaaacctcgtgctcaccgaggaggacgcgaactactggcatatgtaccagaccctcatgggcgactcgacggatggctacgcggggtgtcccggcgtgggtcccacgagtgccgctgagttcctggccgagccgtacatcgcgtatcaggccacgaaggtcctcaagtcgggtccccgaaagggccaagaggagacctactggaccacacgaccgctcgaagaaggcgaggacctgtgggacgggatcgtgtccctgttcaagaaggccgggttgaccgaggaagacgcactggtgcaggcccgcgtggctcgcatcctccgcgcctcggacttcgacttcaaggccaagaccccgatcctctgggagcgcccgccgaaggaggatgtggggaccgaataacccacactaaaggagtcaagcatgtgcttcttcaaagccccgaaggtggaaacgcccaaggtcgaaccaaagccaaccatcaccgagacctcggcacccgagccgcaggccctcgtggtgggcggcgtggacgacaaggcggcatccgctgacggcgaatccaagtccagcaagaagggtacgggctcgctcaagatcgacttgtccccgactgcttctgaatcgggagatgccaatgtcggaaccggcgcgaaccaagccttcaacgtcaaccgtggaggcggcggacccgctcgccccaagaaggctgtcagtaagggtccgacaatgtgacgacctcgggttcatgagcgaggagctagtgaaggccgcgtgggaaggttccccgctggcccagcgagcgttcctcgattgggaatcctactggcaacaccagaagaagttccacgagggcacccagcaggcccagatcgttctcatggacggcgagacctgcgtgggcggcatcgtgctgcacccctgtgaggacgcccaagtaggcgaagccctgctggcgctccacacgttcatccttccagcgtaccgctcggtcgccaacctgaaggcccttcgagagaaggcccggtgggttgcgcaccagtgggacatccgctggctggtctttccacgggggacccctgagggggttctccacaagtatctggaggtttaactatgggtggcatcaagaaggcggtgaccaacatcatcaagaagccgttgcaggacgttggcatcatcaagaacgacccggctcccgaagcggccccggcaccggcccctgctgcacaaccggccccggtgatcgccccggctcccccgcccccggctgccgcaccggctgcgcaagctgctgtgccgaccgatggcggtaacccggaaacccaagccgaggcgcaggcgaaggccaaccgtcgcggcaagaagggcgtgaccatcaaccgcatctcgggcggcggctcgggcctgaacgtctaaggcgatggcgaagcccggtgaccgctcgggcttggacgaggaaggcgcacgcgcgatctaccagcgactcatgaatgatcgcgctccctacgtgacccgtgcggagaagaacgcgcagtacaccatcccgtccctgttcccgaaggcgagcgataacgcctcaaccgattaccccacgccctaccagtctgtcggtgcgcgtggcctcaacaacctcgccgcgaagctggtcctctcgaccgtgcccgtgggcgaacctttccaccgcctcaccatctccgagttcgacatcaaggcgcaaggcgcccagactggcgaggagggctctgtcatggacaaggcgcaagtcggcctgtcgatggtcgagcgcatcgtgacggcgcacggcgaggcagcaggtttgcggccaacggccagcgaactgatgaagcaactcctcgtggccggtaacggcatgttgcacttgcccccgggagaggtggcaaccaagctgtaccgcctgtcgtcctacgtctgcgagcgtgatgcagtgggcaacgtcctgcaaaccatcgcgctggacaagctggcgtatgtggcgctgcccgaggacctgaaaggctccctgccccagcaggactacgagcccaaccaactgatcgagatttacacccactgctaccgcgacctcgaaagcgatggctggctcgaatatcaggaggtcgagggcgaagtggtgaagggctccgagaacacctacccgaaggaagcctccccgtggattcccatccggctctacaagctggacggcgagaactacgggcgctcgttcgtggaggagtacatcggtgacctcgtttcgctggagaacatctcgaagtccatcgtgcagttcgccattgcgtgctccaagattctgttcctcgtcaagccggggtccagcacttcggtgcgccgtgtggcgaaggccaactcgggcgacttcgttccgggcaagaaggaggacatcgaggtcttccagatggagaagttcgctgacttccagatcgtggagaaggtgggcaacaacatagagcagcgcctgtcgttcgcgttcctgctgaatagcgcggtgcaacgctcgggcgaccgggtgacggctgaggagattcggtacgtgtcgcaggaattggaggccacgctcggcggggtctactcagtactggcgaccgaattccaactgccgctggtccgccgctggctggttgacctgcaagccactggcaagattcccgacctgccgaccgaggcgctgaagccccagatcatcacgggcatcgacgcgattggccgaggccaggatcaagccaagctgtccgccttccaagcgatgatcacgccctacgtggaccgtgtatccaaccgcgtggattgggacaacctgctgctgcgcgctgcgaacgcctccgggatcgacccctctgggctcatcctcacggaccagcagatgcagcaacgtgcggcccaagagggcatcacgcagggcatggtccaaggcggcgctgctgctggtcaagccgctggtgccaacctcggcgctggtgctacggacccggaagcgatggccgctgcgatgggctaataacccacactagaggtgacccttcggggtccctcatttcccccacttcaaggagaacacatggaagttactgcaagcgacatctacggctctggcgcaatcgtttcgtctggtgaactgacggacgctgaacgtgccctgctggacaacccggcgagcatccgtgatggcgacgatgtgatcgacattcaggtcgagtccgaggacaagcccgatggtgactccgaggaagtccaactggacacctcgaagggtgaccaagagggtgaccaagaagaagaagtcgaagtggaccccgaaacgggcctgccgaccgacgagaacatccagaagggcatcaacacccagcaggcagcgattggcgaacacgcccagaagatcgcagaagctggtctcgacccggttgctatcgtggacgagtaccaagccaagggcgcgctgtcgagcgccacctacgagtcgctggaaaaggctggctactcgcgggctgctgtggacgcgatcatctcgggccaagaagcccaagcgcaactgttcaaccagtcgatctactccagcgtgggtggtcaggccggcttcacccgcgtggctgaattcgcccgcacgaacgacccggctggtgccaaggcgtacaacgatgcgttcgagcgtggcgatctggccgcgtgcaagtcgctgctgaagtcgttccaagttcagatgggccagaagtacggcaccgcgaacaagggcgtccgtggcgccaagcccgtaacgcagggcaccgtgaaccgtgcgaagccgttcgagtcgcagcaggacatggtgaaggcgatgtccgacacgcgctatggccgcgatgcgaagtacaccgccgaggtcgagcgacgcatcaacgctggctgacgattaacccacactataggacagaggacgcagcaacagcacggcctcgaacctagacatgactggctcgcttcggcgggccttttctttttcagatcaaggagattcacacatggcaactaccgtacaaaatccgggtcaagtcgcaaacgctggcgaccgtctggcactgttcctgaaggtcttcgctggtgaagtcctgaccgccttctcgcgcaccgccaagtcgatggacaagcatatcgtccgcacgatccagtcgggcaagtcggcatcgttcccggtgatgggccgcactgtcggcaagtacctcgccccgggcaactcgttggacgaccagcgcagcgcgattccgcacaacgagaagatcatcgccatcgacggcctgctgaccgctgacgtgctgatcacggacatcgacgacgcgatgaaccactacgatgtgcgcggcgagtactcgaagcaactgggcgaagcactcgctctggctgctgacggctcggtgctggccgaactggccgctctgtcggctgtggccgagaacctgacgggtctgggcgccggtgcgcagatcgaactgaagaccgccacctcggtggccgtggctgacccgaccgttggccaagaaatcctggcctcgctggcgcaggctcgcatggtgctgggcaagaagtacgtcccgaccgctgaccgtgtgttcttcgtgacgccggaagcctactcctcgatcctcgctgccctgatgccgcaatcgagcaactaccacgcgatcatcgacccggaaacgggcaacctgcgcaacatccacggcttcgagatcatcgaagtgccccactttgaactgggtggcgcggatggcaagcacgccttcccgcctgcactggctggcaaggtggtcggtctggccgctcaccgctcggctgtgggcaccgtcaagctgaaggacctcgcactggaacgtgcccgccgtccggaattccaagccgaccagatcatcgccaagtacgcgatgggtcacggtggcctgcgtccggaagcggtctgcgctgtgctgaccaaggctggcgcctaatcccgctggggacccctcgtgggtctccgctggatcgcaaccataaagaacccctgtgggtcccttcggggtctcatgggggttttttcgcaactaggagaactctatggcaaccatcatcactccccgcacggaactcgatgcggtgaacgccatcatcggcgctatcggtgaaggcgtggtgaacaccttggagggggacgctaacgtggacgtgctgaacgcccgtaggctcctcgctgtggtgtccgccgagattcaagataagggctgggtgttcaacactgacgaggcgttcgagcttgtgcccgatacgttctcgaacaagatcgtttggctccccacgtacctccgcgtgatcacccccggggggaccccctacgtgaaccgtgggggcttcgtctatgaccggctgggccgcaccgaccagttcacgggccggatcaccgtcacgatgaccgagcaggttcccttcgaggaactgccgctctgcttccgccagtacatcacgtacaccgccgctgaccgcttcaacgcgcagtactacggtgacccgggcgtggaggccgcgtgcaagcaggccatcgtggagtcgtcacaagcggtgcaggagtacgagattgattatggcgggttcaacctgttcaataacgatccgcactacctcgccaactctgggaggtaacgatgccactcgtcacacaaaccatcaagaacctgaagggcggcatctcgcaacagccggacatcctacgcttccccgaccaaggcgaggcccagatcaacggcttcagttccgaggtcgagggcttgcagaagcggcccccttcggtccacgtcaagcgcctgaccacgttcaccccggggctgaaaccgctggtgaagctgatcaaccgggacgagttcgagcggtacttcgtgtcgttcctccccggtgggtacatctccatcgtggacctcgacgggaacgccaagacggtgaacacccccaacgggaccggctacatcaacagcgcgaatccccgcgaggacctccgcatggtcaccgtggcggactacaccttcatcatcaacaagaaggtgaccccggcgctggacggctcggtggcctacccggggtatcgcaccaacgggcaggcgctggtgaacgtcaagggcgggcagtacagccgcacctacagcatcgagttcaacggcggggtgcaggccagctacacgaccccggacggctcggtggcggcgcacgcggcccagattgacacccagtacatcgcccagcaattgggcaaccagcttgtgtccaacttggggccgagcggctggggcgtggacgtggggccgaactacatcttcatccaagcgcctagctccaacagcgtgtggaacctgaagattcgggacggcttcaacaacggcctgatgaccgggtgcatcttcgaggtgcagcggttcaacatgctgcccgcgcaggcgcgtgatggctacatcgtcaaggtgctgggcgaccccggcagtggctcggatgactactacgcccgcttcgacgtgggccgtggcgtgtgggcagagtgtgccgccccgggctaccaagggacgctggccccgtggtcaatgccgcatgtgctggtgcgcgaggcgaatggcacgttcacgttccgcgagcagacgtggcaggtccgcccaagcggggacattgattcgagcccggagccctccttcgtggggacgcctatctcggacgtgttcttcttccgcaaccggctgggcgtgctggcgggcgagaacgtgatcctgtccgcatcgggcgagttcttcaagttctggcccaagtccgtggtcgctgccgcagacaccgatcccatcgacgtggcggtctcgcacagccgggtctcgatcctgcaccacgcggtcccgttcgcggaggagttgctgctgtggtcggaccagacgcagttcatcctgaagtcggacggcatcctgtccacgaagaccgtcaaggtggacaccgcgaccgagttcgagtcgagcatcgacgcccgcccggtggccgctggccgtggtgtgtacttcgccgccccgcgtgcgagcttcacctcgatccgccgctactacgcggtgcaggacacctcgcaggtcaagaacgccgaggacatctccgcgcacgtcccgagctacgtcccgaacggggtgttcgcgctgggatcgtccacgactgagaacgtggtcacggtgctgaccgagggcgccacgagccgcatctacctctacaagtacctctacttgcaagagcaactggcgcagcagtcgtggtcccactgggacttcggcccgggctccgaggtgctggcctgtgagatggtcggggcggtcatgtacctgatgatcaattccccctcggggacctacctcgaatccatcgagttcacccagaacacgaaggactacgacttcgagccgttccggttgcacatggaccgcaagaagcaagtggagaccctgacgtacaacctcgccacgaaccgcacggtcatctcactggcgaccgagtacggggccacccccgcccgtgggaagtactgggtggtgaccgaggacgggcgcggctatgagttccccgagccggagaccggatgggtagcccaaggcgggacgctggagattcccggcgacctgacctcgaagacgatgctcatcggggaggcgtacacgttcacctacacgatgtccaagctgttgatcaaggtggcagacgcccaaggggttcgctccgaggacgtgggccgcttgcagattcagcgggcatgggtgaactacaacaactccggcccgttcaccgtggatgtgtgtgggaagttcctctacaccatgagcggcaagaagctgggcgcctacgtgctgggcgaggacgcgctggataccggccagttccgattcccggtgatgacggactcgcagcggtgccgcgtgaccatctcgtcggacaatcctggccctgtggcgatgattggcgcgggctggatcggcaggtatttccgccgtgaacaggcgctgtgatggggcctgattaacccacactataggaggggtccctaaggaaaaccattaaaggtaatcctaaagggaccccttcttttctttcaactgagaggagattaccaatcatgcgtttgtatagcgaagacctgaccccgaagggcatcatcgagttgatcgagcaggctctccctgaggataggcgcgagtatgagtgcctgactggcaaccccttcgagtcccttgcggggtccctgatggactacctcgaaagcggcgcccatgcggaggccgtgcgggacccggagggcaacctcgtggctgtcggcgggctggaccatctcggtgtctgctggttcgtcaccacggcccgagtgaggtcccataagggggacttctgcaagatcatcaagcaccgcagggatgtggtccacacggtctacggggtcccctgcgtgaacatcgtcatgatgtccaacgacctgcacgttcggttcctgcgttatctgggggccgagtttatcaacccagtcacgatcaacggcgaggagttccgcacgttcgtgatcaaaccgaggagcgacgatgtgtgaaccagtaaccattggtatgggcatcatggccgtggtcggcatggcgtccgccgcttcggcccagaaggaccaagcagaccagatcggtgatgcgcgtgcggcccaagcggggcaggcacggcaactgatcaagcagatgaactacaaggacggtcaactgaatcaggaggaccgcaacgcctacgagcaggcccagcagcaactcgaaacgaactcgatcaatgccatgcgcaatcgagggatgatcgaggccgcgttcgctgagtctggcgtggagggccggtcggtcgattccgtgatccgcgaggtcaagggccaagacgcccgcgtggccgactcgatccgggccgactacatgaaccagcgccgtggcatccagtctgcgtccgagcagaactacatggagaccactggggccatctccgggcaggccaagattcgcggcccgtccaacgtgtcccagaccctgcaagtcatcaatggcgggatgcagggcgcgcaggcaggcgcctcgatgggcaatgcctattccgcagcaaccgcgccgaagggtacgcccccggccaaaatctaaggaggtcacatgagcgccaatagtgcatacatgatgtggcgccagttctccacgagccctcgcgtgtcgaagggtgtcgaggctccgggctaccgcgcccagtccgtacagaacactgtggacatgcgtggtcggagcactgcggggattctggcggacttcgccaacaacgcctcgcaagcgttcaacaccttcaacaaggtgcagtccgacgaggccaacaccaaggtccaggattggatgaaggacaagacggtcgatgaataccgcgccgagatgaaggccggtaacgtcccgttccaagacgaccaagtggcaatggccgtgctgcacaacaaggcggcatacaccgctgcgttgcaggtcgagcaggacgtggaggaccagattcagcagggcaagttcaaggacttcaacgaggcggacaccttccgcatcaaggccctgaacagcgcccgcgccaagtacgccgagcagttcaaccttaccccggacaacgcggcgttcaaggctggcttcgaccgggaccaagagaagcgccgcgaggtgctgctgcgcctgcaaacggacgtgaccaataagcgcctgcaacaagagggcatgacggtcgcggccacggacctgatcgccccgctgccggaagtgctccagaacttcggtgcggagggtgcggccaagtacatcatgcagaccacggccatgcacgacaagaccggcttggcccgcacggacgctgaccggctgacgctgatcgggaaggcggtcgattccctgtcggcctcgaagggtggcgccgatgtgctcgaacaactgggcgggcagacgatcagctacggcgggcaggacgtgaagctgcgcgacctgatgggcgggggcaagtttgacctcgctgtgattcaggcccgccaagcggagaacgcccgggacggcgagcggttcgtcaagcaggaggccgactattccaactggatggcgaccaaggatgacaccgccgcacagaagcacatcgaaacgctcctgaaggaatcccggggggtccgcacccctgaggtgaaccgagccacggaagtcctcgggttcatcaagaagaagcaggagttcgaggcccagcaggcccgcgagcagtacgccatgcagcttgagaagcagggccgcattcagggcgcagtccggaccctctcgggtgtcctctcggggaccatcacgggcggcgtgtcggctgaccctgacggcctcggtctgaaggaccgcgaggaactggtcgctgccgagaagatgatcgtggacgggctccctgagggtccccagcgggacaccgccatcctgaggctagcctcgacggtccccaatgggtacgccttcaaggccgtgaagggcatcgtggattccagcgaacgcgactgggaactcatgcaggcccagatcgcatccggcaaggcggacgtgaaggttccaccctcggtggagcgggtgcaggcgttcatgaagctgggcgagggttcctcggtgctggctgtgaacaacggcaagcccccggcctactggtcggccatcgaggcaggcacccggatcggcgtgaccccggctgacgtggccgtgagtcaggcagcgtggaaagccctcccggagaaggagcgcaacacgcggatgcaggccctgaccaagccgctggcaaacctgaacgtgccgctgaaccagcgcaacgtggagacattgcagacgttcgcgggccactacatggcgatgggcctgtctgccgagtctgctgcgaagcaggccgagcaggacttccgggaccagaacgaggtcttcggcaagggcaacggcgtggtccacaagtcgttcttccagttcgacggttcgcgtaactcgttcgccgctggccgcgccgcgttcgacggcatccttgccgagacccgcaacggatggggcgtgggcgaggagcgcacgatgctggactacagcccggacagtcagcaggtctcgatccgcaacgtgatgaccggagagtcccggcccgtcacgcaggacgatgtgcgggcgaagtacaagtatgacgctgagaaggccgcgaaggccaacaaggcgaaggtggatgaaaccatcaagcaggagtcccaacgccagaagaagcgttccgaggttaccgccgaggacgtggcagcagcagcaggcgtgggaactgtccgataacctttcaaggagattcacatgacgacaaagtacgacgcgctgatcgagcaatcggcgcaggccaacggtctcgacccggccaagctgagggcgcagattcaagcggagtcgaacttcgacccggctgtggtctccaaagctggcgcagtcggcatcggccagatcatgcccaagtactggatgggtcagcacggcctgaacagcatcgaggacttcaagaacccagacaaggcaatcccggcgatggccgctatcatggcgcagaacgtgaagcaggccggaagctgggagggcggtctggtcctctacaacgcgggcgcaggtaaggggaacaagtacctgaacgccttcaaggccggtcagtatgacctgctgccggaggagacccgtggctacctcgggaagctggccccctcgctggggattgcctcgggaaaagccccgggcgtggtcggcctcagtgatgcccgggttgacctcaacacgcccacacaggtacaggggcaggcactagggcaatacagggacctcaacgccgagcaaggactcggtgacagtttcctgtccggcctcaacgcttctgctattggcaccgctctgcgccgttctgatagcccgatggcttcgctgctgggcggctcgaacccgctctccgaagaagccctgagcaagattgccggggccaacatcggggccagcgggaccaagttcgtgatgcgcaacgccatgaacgagcaacaggtcgatgaactgatcaaccttgcccgggagaaccaagcgagcgccaaccagaagcgaaccctcatgggtgacctttcgtatggcactggcgagatgctgggggaccccgtgacgtatggcacgatggtgatcccggctggggccgctgtgaaggccgggcggctgttctcgaacggtatcgctcgggtggcctctgcgggggccgttgcggctgctgagggcgctgcctcgaacctagtgtcggaagccttccgggaatccaccacgggcaacgaggcggacttcgctggggccatcgcttcgggtgccgccttcgggctgggcctgcacgggctcggcgtggcgggtggctgggcgtaccagaaggccgcgaaccggctgacccgtggtatccaccgggccgaggcaggcgagactgtggccgcgctccgggcggatgggcacacggacgcagtggacccgacgatcttcacgccgatggacatcgacaacgagaccgggatcaagtggaagcaaaccatcgaccggggcgagttcaccgccgcagaagcggaagcccgccgtggcccgaactaccagccgggtcaagtgccggtgctcctgaagacccccgctggggacaccctgcacacgcccacgggcatccagttctcgtcggccaacccactgaacccgggccgagtcgaggccaacctcgcaggcgccccgctaccgttcgatggggtggagattggggacgttctgagccgcaccgagaaccaatccctgaaggacctgttctggaacctcggacggtccacccgggggtacaccgatgggtcctccggcaagttcggcgtgacgggccaggatgtcgcccagaacatgaacggtcggttccacgactaccagtggcaactcgatgcggcccgcacggaagcccttgcggagccccagtgggtcaacgccccggggtcagcccgaggggtgcgccaagcgttcaacgagaagattcaacgggcgatgttcaagcaggacccctctgggctctcgaaggccgaacgcaaggtgtacgacttgcgcaccgagttctaccgcgatctggggcagcagcaggtcaccccgggggcacggtggggcgtggatgcgcccagcctgctggacgaggcgagcttcaagaagaactacggcgccccgatcatctatgacgatgtgaaggtgcgcgccctgacggaccagatcggtgcggacgccttgcaggacctcgtggcccgcagcttcatcggctcctacctgtcgaacccggaggtgcgtaaggccgtgatcgagcgcatcgcgcaggacgcagcgcagggcatccagcgggacttccggcaactggcgcacgacatcgcctacgggattgtgaagtcaggcgacccgctggacggcgtgggcgtgtctcacctgaaccgcatcatggattcgggcgtgggccatgccgactccaccccgggcttccgcaagcaacgcaacccgttcggccatgactacgagattgaggtccccggccagcagggcaagttcagcgtggcggacctgttcagctacgacacggacctgatcgacacggggtacttcaaccgcgtgcgcggcgatgtgtctctgagtgtgggcgctggcgctgggctgtcggacgtgacggacatcatccgttcggcgcgggagttcgcaggcgcggtggccccggagcagaaggccgctgtggacgctgccgagatgctggtcaaccaactctacggggtcggcatgaacagcgactgggcgcggcttcgtgcggtcgagggcatcgtcaagaacctcgccttcatgaagtcgtccgcgttcatgggcctgtccaactacaccgagattgcggcaggcatccgcgagcacggcatctcgtttgcggccaagtctatcccgggcgtgggcaagctgttctcgaacctgatgaacggcaagaccacggaagcgaacatgcgactggcgcagggtctggtctggggccgggaactggacaaggcgatcatcccctcgttcagcgaggccatcgagcgcagcgtggacaacctgagtgaccgcatcggggagggcatcaccgcctccgcactgggcgggatcaagggcgcgaccgaggctgctgccgacaagtggtgggcgaccaagttcctgcgggcgaccagtggtcgcatcgtggagactgcgcgggccgagttcttcgcggacctcgcccgtggggcgcacactggcaagtcgagcttcgccaacgtgaccaaggccaaggccgcgtccgtgagtcccgagcaactgaacggggtgctggacctgctgcgtgaatcgacccagatcgtggacggtgaactgaaggtgacgaacccgaaggcactgaccgacgatccccgggccgctgcactgcgccgctatgggcagtactggtcggagcgggtgatccagcagaacacggtctcgtcaaccttccgctgggccggtatgccggtggtcgggatgctgtcgcagttccagtcgttcgtgatgcgctcggtgaatgccaagctgatccgagggaccgctgcgacgttccgcgatggggatgtgggtcagggcatcgacaccttcatcctcgggccggtgctggcggggctcggctacgccgggatgacgtacctgcgcgcccagaagttcagcaacgaggcagaccgccagaagtacctcgaagaaaacctcggggagaagggcgactactcgatgctggtggcgggctccctgaagcgttccgccgcgttcggcgccatcggcaacctgcacgacaccgtggtgtccacccctctggtggcccagtgggcgcccgactacagcgccaagtatgcgggccttgggcggacctcacaggaggccaagctgaggcgtgaccggcagagtgcccagcagggcgcccctgtggggaacctcctcggcggcatcgtgcagaacgccccggctgtgaagctggcggattccctcgcggggctcgcgtgggcacccgtggatcgtctgacgaccccggctgactcctttgacgaggaccgctggcataagggcgtggtgcgctccatgaagggcctcgtgcccaacgatccggtctcccagcgggcattccaagagtggatcgctgacccgtactgaccgggagattaacccacactgtaggacataggctggcccttcggggctggcctttttttcgttaaccgataaggaggacacacatggctgcaccaacaaccgtgaaggtctatccgctgaataacgttgtcaaggacttccccattgacttcgattatctggcccgtgagttcgtcgtggtgaccctgctggggaacggccgccgcgaactggtccagaacagcgagtacaccttcctgagtgccacgcagattcgcacgaccgccacttggggtcccgcgcagggctacgacaacatcgaggtccgccgcgtgaccagcgccgagaaccggctggtgacgttcaatgacgcctcgatcctgcgggccgctgacctgaacctcgcggaactgcaaacggtccacatcgcacaggaggcccgcgatctggtctcggatgcgctgggtacgaatgacgatggcaacctcgatgcacgcaaccgccgcatcgtgaacctcggcaacccggtcgatccgcaggacgcgatgaccaaggactactacgactcgcggctcggtgaggccaagacgtggcgcgacgaaacgttaactgcccgcgatgcgaccttcaccgcccgggacaccacgctaggctaccgcgacacgaccctgacgtacaagaacgctgccgaggcggcgcgtgacgctggctttgcggcccgtgacgtggcactcgctgcgcgggacaccacgctcggctaccgcgacacggccaaccaacaccgcatcgacgcgcaagcggcccgcgatctggcgctccagtatcgcaacgaggccgaggcgttcaagaacactgccagcgacaaggcagacatcgtgctgaccatcgaggccacggccaactctgctaaggcggaagcccaagcggcgaccgctgccacgaccggcctgatggacaacgacctgcaactgttcgtgaacggcacccagcgccgcgacatcaaggtcaaggcgggcaagcatgacgcccaaggcgttcaacgtgcaggtgtccgcttcactgacgtggacaccatcgctgtcgagcggtacaaccagaccacgggcgtgatcatcgacactccggtgaagatcgaccaagcgaccggggatgtcgtgctcgcgtccaagaagatcaagggcgtgaccactcccacggccaacgacgaagcggccaacaagagctacgcggacaccaagctgccgttcgtcggaggcaccctaaccgggtctctgttcgtgagtggcgcgaccgacaacgctggggccttccgggtcacggctgacctcggcggtgcgtgggtggattggaacgcggcacgcaagtacccgctccagatcgactgccccgcctcctccgctgcctacggcggcatccggtggacacgctggggcgcccgccatatcgccgcaattgacgccttcgacaacaccggaggcgccccagtaatcgtgatgcaccttggcgaccagaacaatgcgtggtcgttctacaaggacaacatctaccgggggtacaacggcggctacgtgtggggctcgtggaacttcaacccggccagcaaggcgaacgccgactggatcaccgatgttggcatcgaggggaacgacatccgccgcccctacgttcgacgcgccagcgatggctacatcgtccggatgccccgcaacttctccggcaacgcgctggaggtgggctgggatggtggcctgcggtggtacgtggatggggtccaccaaggcgcgatggtcagcgatacgaactggcgcggcgcgttccacaacgacgcgggccggatcggacgcggcggtgactggtccatcggcaacggctacttcgacgtggtgatcgacggggtttcctacggggtccccttcaatccctcggatgctcgccgcaaggagaacatcgtgccgtccaccgaggacgccatcgagaaggtgaagcagatcaagttctactccttcaacttcaaggcggatggcctgatggactccaagaaggtccaccagactggcttcatcgcgcagcagcttcagggtgttgatccggcgctggtgatcggtgacgacaacccggatatgaccctctcgcctgacgtgaactcgctcctgtcgctggccctgaagtccatccagcaactcgacgcgaagatcgcagtgctggaggagaccatcctggccctcaaaggtaacccctgatgagaccccagcgcgtccgtatcaatgggcgccgctggacccttgagtaccccgaagcgatagacgacgagggcacctacgggatcacctactacgacacccacacgatccaagtgcgggacggtctggctcccatcgaggaggccgacacggtgatccatgaagtgctccacgccctcatcgcttcgatgggcctcacggtgcccgacgaggagcccatcgtgcgcgccctcgcctcggggctcaccggggtcctagcggataaccccacgctcctgaagcacctgaacgggtgcctgaaggggtcccccaagaaggagaagtaatggtcaatctcgacgttcaaaacgggatcgtccagtttgccccgcccgccgtggtagccggagaggttgcctcgcgggtgctgggcctgacgatcaatgagtggttctacatcgtggcgatcatctgcatgttgatcagcaccgtcacgacatccctcgtggcgttcattaagtctcgcaagaaggatgacaacaagaaggaggacgaatgagcaagctagaagaactgctttcccagattcacctcgaagtcgcgcagcagatgcttgccgacctgcgggacgaggagaagcgcacccccgcgctctacaacgcgatcatcaagttcctgaaggacaacggcgtggaaatcgagccgaagtccccggaggagaagaaccgagaggccgctgagaagtccagcgcggcccagatcgcggccctgtcccaagaactcccgcccgtgctggcggatgacgagggcttcgcccaattccattaacccacactagaggagcttccctatgtccatcgagcaactgaaggcggacacccgggaagccctgctgcaaaagcgtatgcgcgacgacttccgagtcttcgtgtggttcgtctggaaggtgatcaacctcccgaatcccacggcgattcagaacgacatggcgctcacgctccagaacccgccctcgcggcgcttcatcttgcagggtttccgtggtgtggctaagtcgttcatcacctgcgcctatgtggtctggcgcttgtggcgcgacccgcaactcaaaatcatgatcgtgtcggcctcgaaggaacgcgccgatgcgaactcgcagttcatcaagaagatcatctcggaaatcgagttcctgcatcacctcaaggcgaagccgggacaagtagacaccgtggtgaaattcgacgtaggcccgaagctgcctgaccactcgccctcggtgaagtcggtcggtatcacgggccagcttaccggctcccgcgctgacatcatcatcgcggatgacgtggaggtcccgggcaacagttcgacccaaggcgcccgcgacaagctgtttgaactcgtcaaggagttcgacgctatcctgaagccgggtgggacggtcatctatctgggcacgcctcagaacgagatgtcgctctacaacgaactgctgaaccgtggctacaccacgctgatctggcccgcccggtatccgcgtgacgagaagcagcgggccaactacggcacccgcctcgccccgtatctcgcggagcggtacgacgctgacccggatggcctcgcgtggaagcccacggacccggaacgctttgacgagaaggacctgcttgaacgtgaggtctcctacggcaaggctggcttcgcgttgcagttcatgctcgacacctccctgtcggacgccgagaagtacccgctccggttgcgcgacatgatcgtcggcctgttccccaaggagcgagccccaatggcgcacgactggcttcctggccctaccttggagcttggcaaccttccgcagatcgggctcaagggtgaccgctactacggcgcctatggctcgtccaaggagatgtccaactacttcggcaaggttctcgccatcgacccctcggggcgcggcaaggacgagaccggctatgccgtgatgtacttcctgaacggctacctctatctgatggaggtgggcggcttccggggcggctacgaggactcgacgctggaggaactggcgaaggtcgccaagaagtggaacgtgaacgatgtcatcatcgagggtaacttcggggacggcatgtacctgaagctgtttacgcccttcctgcaccgcatccacaagtgctcggtcgaggaaatcaagtcggtcggccagaaggaggtccggatcgcggacgtgctggagccgatcttcggcgcccatcggttctgcgtgagcgagtccgccatcgagcacgactacgcgaccgccaaggacgtggacggcaagttcgacccgaagtactcgtgcttctaccagatgtcccgcctgacccgcgaacgcggcgcgctggcccatgacgaccgcttggacgctgtggcgatggccgctgccttcttcgtggagcgcatggacctcgacgcggccaagcagattcaggaggccaccgaggagttcatcgcggcccacatggaggaccccttgaggaccggcgagcacctgacgcggtacatcgacgggggtgacgggatggtgatcctgtcgagcgaggacccgagtgacgactactggggcgacctcaacatgcttgacggatgaccatataggtgcctgagagggttccggctacagatcatgacaaattgatctaaggctggaacccttgctggtggccggtctccgggtgtgtcaagaactatttcgattaacccacactatagagggggagccccggtaaggtatatacagtggccctcccaacatgagaccagcaggcactagaaggagccctctagggttcccgctgggggcctatgctgatctgctctctgtgcaatatcaatcattggatcaacatcataaaggaggtgatcatggacaaggtgaagactgtgttgaaggctgtcctgcgtgacaagcggttttggggcctcgttgctgtggccgtggctggcttgggcgtcagtgtcgtacctgctggcaccatcgaagctgttggctgcgctctggctggcggctgtgtgtgaagcggtggcgcgtgctgccgcccggaaggttcgctgaatcgctcaccttccgggaattacaagtgggatgaactggaggggaccctctagtgtcccatcttgtgtccctgctgtggctgtccttgtggtgaccctgcgggggaacacctgaggggccaggacctgagggaccccaaaaggtgtacaggtaggaaaaatctgagagggtatctcatagaatgagccgctgggattacccccatagggggtccacttgggccactggggcaggcaggaggcccccccgggtgcccctctcggcactccctgaggggaaccggcagggtcaacccactggatttaacataatggaccttatgcgcaatccctataggtcacattgagggtactgataggggctctctatgggctcctctggggtgataggagggctctatgggtttcccta